(19) United States
(12) Patent Application Publication
(10) Pub. No.: US 2004/0243227 A1
(43) Pub. Date: Dec. 2, 2004

(54) Delivery devices and methods for heart valve repair

(75) Inventors: Niel F. Starksen, Los Altos Hills, CA (US); John To, Newark, CA (US); Rodolfo A. Morales, Los Gatos, CA (US)

Correspondence Address: James Hann, Haynes Beffel & Wolfeld LLP, 751 Kelly Street, P.O. Box 366, Half Moon Bay, CA, 94019, US

(73) Assignee: Guided Delivery Systems, Inc., Los Altos, CA

(21) Appl. No.: 10/792,681

(22) Filed: Mar. 2, 2004

Related U.S. Application Data
(63) Continuation-in-part of application No. 10/741,130, filed on Dec. 19, 2003.
(63) Continuation-in-part of application No. 10/656,797, filed on Sep. 4, 2003.
(63) Continuation-in-part of application No. 10/461,043, filed on Jun. 13, 2003.
(60) Provisional application No. 60/524,922, filed on Nov. 24, 2003.
(60) Provisional application No. 60/462,502, filed on Apr. 10, 2003.
(60) Provisional application No. 60/459,735, filed on Apr. 1, 2003.
(60) Provisional application No. 60/445,890, filed on Feb. 6, 2003.
(60) Provisional application No. 60/429,288, filed on Nov. 25, 2002.
(60) Provisional application No. 60/388,935, filed on Jun. 13, 2002.

(51) Int. Cl. A61F 2/24

(52) U.S. Cl. 623/2.11; 623/2.36

(57) ABSTRACT

Devices, systems and methods facilitate positioning of a cardiac valve annulus treatment device, thus enhancing treatment of the annulus. Methods generally involve advancing an anchor delivery device through vasculature of the patient to a location in the heart for treating the valve annulus, contacting the anchor delivery device with a length of the valve annulus, delivering a plurality of coupled anchors from the anchor delivery device to secure the anchors to the annulus, and drawing the anchors together to circumferentially tighten the valve annulus. Devices generally include an elongate catheter having at least one tensioning member and at least one tensioning actuator for deforming a distal portion of the catheter to help it conform to a valve annulus. The catheter device may be used to navigate a subannular space below a mitral valve to facilitate positioning of an anchor delivery device.

### CROSS REFERENCE TO RELATED APPLICATIONS

The present application is a Continuation-in-Part of U.S. patent application Ser. No. 10/741,130 (Attorney Docket No. 016886-001320), filed on Dec. 19, 2003, which is a Continuation-in-Part of U.S. patent application Ser. Nos. 10/656,797 (Attorney Docket No. 16886-001300), filed on Sep. 4, 2003, and 10/461,043 (Attorney Docket No. 16886-000310), filed on Jun. 13, 2003, the latter of which claims the benefit of Provisional Application Nos. 60/388,935 (Attorney Docket No. 016886-000300US), filed on Jun. 13, 2002; 60/429,288 (Attorney Docket No. 016886-000700US), filed on Nov. 25, 2002; 60/445,890 (Attorney Docket No. 016886-000800US), filed on Feb. 6, 2003, and 60/462,502 (Attorney Docket No. 016886-001100US), filed on Apr. 10, 2003, the full disclosures of which are all incorporated herein by reference.

The present application claims the benefit of Provisional Application Nos.: 60/459,735 (Attorney Docket No. 16886-000900US), filed on Apr. 1, 2003; 60/462,502 (Attorney Docket No. 16886-001100US), filed on Apr. 10, 2003; and 60/524,622 (Attorney Docket No. 16886-001310US), filed Nov. 24, 2003, the full disclosures of which are hereby incorporated by reference.

### BACKGROUND OF THE INVENTION

1. Field of the Invention

The present invention relates generally to medical devices and methods. More particularly, the invention relates to devices, systems and methods for enhancing cardiovascular valve repair, especially the repair of heart valves such as the mitral and tricuspid valves.

In recent years, many advances have been made to reduce the invasiveness of cardiac surgery. In an attempt to avoid open, stopped-heart procedures, which may be accompanied by high patient morbidity and mortality, many devices and methods have been developed for operating on a heart through smaller incisions, operating on a beating heart, and even performing cardiac procedures via transvascular access. Different types of cardiac procedures, such as cardiac ablation techniques for treating atrial fibrillation, stenting procedures for atherosclerosis, and valve repair procedures for treating conditions such as mitral valve regurgitation have experienced significant technological advances. In implementing many minimally invasive cardiac surgery techniques, especially beating-heart techniques, one of the most significant challenges is positioning a treatment device (or multiple devices) in a desired location in or around the heart for performing the procedure. Another challenge, once a device is positioned, is to effectively deploy a given treatment into or on the target cardiac tissue.

One type of cardiac surgery which may benefit from less invasive techniques is heart valve repair. Traditional treatment of heart valve stenosis or regurgitation, such as mitral or tricuspid regurgitation, typically involves an open-heart surgical procedure to replace or repair the valve. Valve repair procedures typically involve annuloplasty, a set of techniques designed to restore the valve annulus shape and strengthen the annulus. Conventional annuloplasty surgery generally requires a large incision into the thorax of the patient (a thoracotomy), and sometimes a median stemotomy (cutting through the middle of the sternum). These open heart, open chest procedures routinely involve placing the patient on a cardiopulmonary bypass machine for sustained periods so that the patient's heart and lungs can be artificially stopped during the procedure. Finally, valve repair and replacement procedures are typically technically challenging and require a relatively large incision through the wall of the heart to access the valve.

Due to the highly invasive nature of open heart valve repair or replacement, many patients, such as elderly patients, patients having recently undergone other surgical procedures, patients with comorbid medical conditions, children, late-stage heart failure patients, and the like, are often considered too high-risk to undergo heart valve surgery and are relegated to progressive deterioration and cardiac enlargement. Often, such patients have no feasible alternative treatments for their heart valve conditions.

To obviate this situation, a number of devices and methods for repairing cardiac valves in a less invasive manner have been described. Some devices provide for heart valve repair through minimally invasive incisions or intravascularly, while others improve upon open heart surgical procedures on beating hearts, stopped hearts or both. As mentioned above, difficulties in performing minimally invasive intracardiac surgery include positioning a minimally invasive treatment device in a desired location for performing a procedure and effectively deploying a given treatment into or on the target cardiac tissue. In heart valve repair procedures, for example, it is often essential for a physician to secure one or more treatment devices to valve annulus tissue. Annular tissue tends to be more fibrous than surrounding muscular or valve leaflet tissue, thus providing a more suitable location for securing such treatment devices, such as anchors, to treat a heart valve. Positioning an anchor deliver device in a desired location adjacent the annular tissue may often be challenging, especially in an intravascular procedure when visualization of the location is limited.

Devices and methods that address these difficulties are described in U.S. patent application Ser. Nos. 60/445,890, 60/459,735, 60/462,502, 60/524,622, 10/461043, 10/656797 and 10/741,130, which were previously incorporated by reference. For example, these references describe devices and methods for exposing, stabilizing and/or performing a procedure on a heart valve annulus, such as a mitral valve annulus. Many of the devices and methods previously described by the inventors have been found to be highly effective, but improvements are still being sought.

Therefore, it would be beneficial to have improved methods, devices and systems for enhancing heart valve annulus treatment procedures. Ideally, such methods, devices and systems would facilitate positioning of one or more devices in a left ventricle or elsewhere for performing a procedure on a heart valve annulus, visualizing the annulus and/or the like. Additionally, such methods, devices and systems would ideally be introduced intravascularly. At least some of these objectives will be met by the present invention.

2. Description of the Background Art

Published U.S. application Ser. No. 2002/0156526 describes a catheter-based method for performing annuloplasty. Published U.S. application Ser. No. 2002/0042621 describes a heart valve annuloplasty system with constrictable plication bands which are optionally attached to a linkage strip. Published U.S. application Ser. No. 2002/0087169 describes a remote controlled catheter system which can be used to deliver anchors and a tether for performing an annuloplasty procedure. Other patent publications of interest include WO01/26586; US2001/0005787; US2001/0014800; US2002/0013621; US2002/0029080; US2002/0035361; US2002/0042621; US2002/0095167; and US2003/0074012. U.S. patents of interest include 4,014,492; 4,042,979; 4,043,504; 4,055,861; 4,700,250; 5,366,479; 5,450,860; 5,571,215; 5,674,279; 5,709,695; 5,752,518; 5,848,969;5,860,992; 5,904,651; 5,961,539; 5,972,004; 6,165,183; 6,197,017; 6,250,308; 6,260,552; 6,283,993; 6,269,819; 6,312,447; 6,332,893; and 6,524,338. Publications of interest include De Simone et al. (1993) Am. J. Cardiol. 73:721-722, and Downing et al. (2001) Heart Surgery Forum, Abstract 7025. All of the above cited references are hereby incorporated by reference in the present application.

### BRIEF SUMMARY OF THE INVENTION

Devices, systems and methods of the present invention are generally used to facilitate transvascular, minimally invasive and other “less invasive” surgical procedures, by facilitating the delivery of treatment devices at a treatment site. “Less invasive,” for the purposes of this application, means any procedure that is less invasive than traditional, large-incision, open surgical procedures. Thus, a less invasive procedure may be an open surgical procedure involving one or more relatively small incisions, a procedure performed via transvascular percutaneous access, a transvascular procedure via cut-down, a laparoscopic or other endoscopic procedure, or the like. Generally, any procedure in which a goal is to minimize or reduce invasiveness to the patient may be considered less invasive. Furthermore, although the terms “less invasive” and “minimally invasive” may sometimes be used interchangeably in this application, neither these nor terms used to describe a particular subset of surgical or other procedures should be interpreted to limit the scope of the invention. Generally, devices and methods of the invention may be used in performing or enhancing any suitable procedure.

The present application typically describes devices, systems and methods for performing heart valve repair procedures, and more specifically heart valve annuloplasty procedures such as mitral valve annuloplasty to treat mitral regurgitation. Devices and methods of the invention, however, may be used in any suitable procedure, both cardiac and non-cardiac. For example, they may be used in procedures to repair any heart valve, to repair an atrial-septal defect, to access and possibly perform a valve repair or other procedure from (or through) the coronary sinus, to place one or more pacemaker leads, to perform a cardiac ablation procedure such as ablating around pulmonary veins to treat atrial fibrillation, and/or the like. In other embodiments, the devices and methods may be used to enhance a laparoscopic or other endoscopic procedure on any part of the body, such as the bladder, stomach, gastroesophageal junction, vasculature, gall bladder, or the like. Therefore, although the following description typically focuses on mitral valve and other heart valve repair, such description should not be interpreted to limit the scope of the invention as defined by the claims.

That being said, the present invention generally provides devices, systems and methods for enhanced treatment of a cardiac valve annulus such as a mitral valve annulus. Methods generally involve contacting an anchor delivery device with a length of a valve annulus, delivering a plurality of coupled anchors from the anchor delivery device to secure the anchors to the annulus, and drawing the anchors together to circumferentially tighten the annulus. One device generally includes an elongate catheter having a housing at or near the distal end for releasably housing a plurality of coupled anchors. The device may be positioned such that the housing abuts or is close to valve annular tissue, such as at an intersection of the left ventricular wall and one or more mitral valve leaflets of the heart. Some embodiments include self-securing anchors, which may change from undeployed to deployed configurations. Anchors may be drawn together to tighten the annulus by cinching a tether slidably coupled with the anchors and/or by a self-deforming member coupled with the anchors. Another device includes a steerable guide catheter for helping position the anchor delivery device for treating a valve annulus.

In many cases, methods of the present invention will be performed on a beating heart. Access to the beating heart may be accomplished by any available technique, including intravascular, transthoracic, and the like. Intravascular access to a heart valve may be achieved using any suitable route or method. To perform a procedure on a mitral valve, for example, in one embodiment a catheter may be advanced through a femoral artery, to the aorta, and into the left ventricle of the heart, to contact a length of the mitral valve. Alternatively, access may be gained through the venous system, to a central vein, into the right atrium of the heart, and across the interatrial septum to the left side of the heart to contact a length of the mitral valve. In either of these two types of intravascular access, the catheter will often easily be advanced, once it enters the left side of the heart, into a space defined by the left ventricular wall, one or more mitral valve leaflets, and chordae tendineae of the left ventricle. This space provides a convenient conduit for further advancement of the catheter to a desired location for performing mitral valve repair. In alternative embodiments, a catheter device may access the coronary sinus and a valve procedure may be performed directly from the sinus. Furthermore, in addition to beating heart access, methods of the present invention may be used for intravascular stopped heart access as well as stopped heart open chest procedures. Any suitable intravascular or other access method is contemplated within the scope of the invention.

In one aspect of the present invention, a method for advancing one or more devices into a left ventricle of a heart to contact a mitral valve annulus involves: advancing a steerable guide catheter into the left ventricle and around at least a portion of the mitral valve annulus; passing a guide sheath over the steerable guide catheter; withdrawing the steerable guide catheter out of the guide sheath; and advancing one or more devices through the guide sheath to contact the mitral valve annulus. In some embodiments, the steerable guide catheter is advanced through an aorta into a space in the left ventricle formed by a left ventricular wall, at least one mitral valve leaflet and chordae tendiniae of the heart.

Some embodiments of the method further include deforming a flexible distal portion of the steerable guide catheter to conform the distal portion to the mitral valve annulus. For example, in some embodiments deforming the flexible distal portion comprises applying tension to at least one tensioning member to cause at least one bend in the distal portion. Some embodiments further involve, before advancing the steerable guide catheter, advancing a shaped guide catheter through the aorta to a position within or adjacent the space in the left ventricle, wherein the steerable guide catheter is advanced through the shaped guide catheter. In such embodiments, deforming the flexible distal portion may optionally further involve passing the distal portion through at least one bend in the shaped guide catheter. For example, passing the distal portion through the shaped guide catheter may include passing the portion through a first bend to direct it approximately into a plane with a plane of the mitral valve annulus and passing the portion through a second bend approximately perpendicular to the first bend and having a radius of curvature approximately the same as a radius of curvature of the mitral valve annulus. In some embodiments, applying tension to the at least one tensioning member may cause the flexible distal portion to continue to bend in an arc with a radius of curvature approximately the same as the radius of curvature of the mitral valve annulus. In some embodiments, tension may be applied to two tensioning members to articulate the flexible distal portion in at least two directions.

In alternative embodiments, deforming the flexible distal portion may comprise expanding a shaped expandable member to deform the distal portion. Alternatively, deforming the flexible distal portion may comprise introducing a fluid into a lumen of the distal portion. In yet other embodiments, deforming the flexible distal portion comprises releasing a shape-memory material from constraint. In these and other embodiments, deforming the flexible distal portion may involve articulating the distal portion in at least two directions. Some embodiments may also optionally involve comprising locking the shape of the flexible distal portion.

Some embodiments of the method further comprise urging the steerable guide catheter against the mitral valve annulus. In some embodiments, for example, urging the steerable guide catheter comprises expanding an expandable member coupled with the steerable guide catheter within a space in the left ventricle formed by a left ventricular wall, at least one mitral valve leaflet and chordae tendiniae of the heart. In other embodiments, urging the steerable guide catheter comprises applying an attractive magnetic force between a first magnetic member coupled with the steerable guide catheter and a second magnetic member disposed within a coronary sinus of the heart. These or other embodiments may optionally further include urging the guide sheath against the mitral valve annulus. Again, urging the guide sheath may involve expanding an expandable member coupled with the guide sheath within a space in the left ventricle formed by a left ventricular wall, at least one mitral valve leaflet and chordae tendiniae of the heart. Alternatively, urging the guide sheath may comprise applying an attractive magnetic force between a first magnetic member coupled with the guide sheath and a second magnetic member disposed within a coronary sinus of the heart.

In some embodiments, a delivery device is advanced through the guide sheath for contacting and delivering a therapy to the mitral valve annulus. In one embodiment, the delivery device comprises a device for delivering coupled anchors to the mitral valve annulus. In such an embodiment, the method generally includes delivering a plurality of coupled anchors from the anchor delivery device to secure the anchors to the mitral valve annulus and drawing the anchors together to circumferentially tighten the annulus. The method my optionally also include expanding an expandable member coupled with the anchor delivery device to urge the delivery device against the length of valve annulus. Alternatively, the method may include applying an attractive magnetic force between a first magnetic member coupled with the delivery device and a second magnetic member disposed within a coronary sinus of the heart to urge the delivery device against the length of valve annulus.

In one embodiment, the anchors are delivered from the anchor delivery device through a distal portion of the guide sheath to attach the distal portion to the mitral valve annulus. In this embodiment, the distal portion of the guide sheath is detachable from a proximal portion of the guide sheath to remain attached to the annulus. Some embodiments may also include cinching the attached distal portion of the guide sheath to circumferentially tighten the valve annulus. In an alternative embodiment, the anchors are delivered from the anchor delivery device through a detachable, biocompatible strip coupled with the anchor delivery device to attach the strip to the mitral valve annulus. Some embodiments include cinching the attached strip to circumferentially tighten the valve annulus.

Some embodiments of the method include contacting a stabilizing member with the valve annulus on a side of the valve opposite the anchor delivery device and applying force to the stabilizing member to immobilize the annulus between the stabilizing member and the anchor delivery device to facilitate delivery of the anchors. Alternatively or additionally, one embodiment may include stabilizing the annulus with the anchor delivery device prior to delivering the anchors. In some embodiments, the delivering and drawing steps cause a first length of the valve annulus to be tightened, and the method further includes contacting the anchor delivery device with a second length of the valve annulus; delivering a plurality of coupled anchors from the anchor delivery device to secure the anchors to the second length of the annulus; and drawing the anchors together to circumferentially tighten the second length of the annulus.

In other embodiments, the method may include delivering energy from the delivery device to tighten the valve annulus. For example, delivered energy may include but is not limited to radio frequency, ultrasound, microwave or laser energy. Other embodiments may include delivering at least one pharmacological agent from the delivery device to tighten the valve annulus. In yet other embodiments, a visualization device is advanced through the guide sheath for enhancing visualization of the mitral valve annulus. For example, the visualization device may include but is not limited to an ultrasound device, a camera, an endoscope or a fiber optic device.

In various embodiments, any of the method steps described above may be performed while the heart is beating. Alternatively, embodiments may be performed on a stopped heart.

In another aspect of the invention, a method for advancing one or more devices into a left ventricle of a heart to contact a mitral valve annulus comprises: advancing a shaped guide catheter through an aorta into the left ventricle; passing a steerable guide catheter through the shaped guide catheter and around at least a portion of the length of the mitral valve annulus; passing a guide sheath over the steerable guide catheter, within the shaped guide catheter; withdrawing the steerable guide catheter out of the guide sheath; and advancing one or more devices through the guide sheath to contact the mitral valve annulus. Various embodiments of this method may include any of the features or steps described above.

In another aspect of the invention, a method for treating a mitral valve annulus of a heart includes: advancing a steerable guide catheter into a left ventricle of the heart and around at least a portion of the mitral valve annulus; passing a guide sheath over the steerable guide catheter; withdrawing the steerable guide catheter out of the guide sheath; advancing an anchor delivery device through the guide sheath to contact the mitral valve annulus; delivering a plurality of coupled anchors from the anchor delivery device to secure the anchors to the mitral valve annulus; and drawing the anchors together to circumferentially tighten the annulus. Some embodiments further include, before advancing the steerable guide catheter, advancing a shaped guide catheter through the aorta to a position within or adjacent the space in the left ventricle, wherein the steerable guide catheter is advanced through the shaped guide catheter. Various embodiments of this method, too, may include any of the features or steps described above.

In another aspect of the present invention, a device for facilitating placement of one or more devices in contact with a heart valve annulus comprises: an elongate catheter body having a proximal portion and a distal portion; at least one tensioning member coupled with the proximal portion of the catheter body and extending to the distal portion; and at least one tensioning actuator coupled with the proximal portion and the tensioning member for applying tension to the tensioning member to deform the distal portion to allow it to conform generally to a shape of the valve annulus. Typically, the catheter body may be advanced intravascularly to the heart to contact the annulus. In some embodiments, for example, the catheter body may be advanced through an aorta and into a left ventricle of the heart to contact the valve annulus.

In some embodiments, the proximal portion of the catheter body is relatively stiff compared to the distal portion. Also in some embodiments, the catheter body further comprises a rounded, atraumatic distal tip. The catheter body may optionally further include at least one radiopaque portion at or near the distal tip for enhancing visualization. The catheter body may also include at least one lumen extending through the proximal and distal portions for passing one or more fluids.

In some embodiments, the at least one tensioning member comprises two tensioning members, allowing the distal portion to be deformed in at least two different directions. The at least one tensioning member may be made of an suitable material, such as but not limited to Nitinol, polyester, nylon, polypropylene and/or other polymers. The at least one tensioning actuator, in some embodiments, comprises a knob coupled with the tensioning member, wherein turning the knob in one direction applies tension to the tensioning member to deform the distal portion, and wherein turning the knob in an opposite direction releases tension from the tensioning member to return to the distal portion to a less deformed configuration.

Some embodiments of the device further include at least one urging member coupled with the distal portion of the catheter body for urging the distal portion into contact with the valve annulus. For example, the at least one urging member may comprise an expandable member for expanding within a space in a left ventricle formed by a left ventricular wall, at least one mitral valve leaflet and chordae tendiniae of the heart. In an alternative embodiment, the at least one urging member comprises at least one magnet coupled with the distal portion for applying attractive magnetic force between itself and an oppositely charged magnet disposed in a coronary sinus adjacent the valve annulus.

Some embodiments further include a housing coupled with the proximal end of the catheter body, wherein the tensioning actuator is coupled with the housing. Optionally, the housing may further comprise at least one fluid inlet port in fluid communication with at least one lumen in the elongate shaft for introducing one or more fluids into the lumen(s).

In another aspect of the present invention, a system for facilitating placement of one or more devices in contact with a heart valve annulus includes: a shaped guide catheter having at least one curve toward a distal end for positioning the distal end in a position below the mitral valve; a steerable guide catheter passable through the shaped guide catheter and having a steerable distal end for advancing around a length of the valve annulus below the mitral valve; and a guide sheath passable over the steerable guide catheter through the shaped guide catheter, wherein the one or more devices are passable through the guide sheath to contact the mitral valve annulus. Generally, the shaped guide catheter, steerable guide catheter and guide sheath may have any of the various functions and features described above, in various embodiments.

In one embodiment, for example, the shaped guide catheter includes a proximal curve approximately perpendicular to a central axis of the shaped guide catheter for bringing the distal end of the catheter into a plane approximately parallel with a plane of the mitral valve and a distal curve having a radius of curvature approximately the same as a radius of curvature of the mitral valve annulus. In one embodiment, the steerable guide catheter comprises: an elongate catheter body having a proximal portion and a distal portion; at least one tensioning member coupled with the proximal portion of the catheter body and extending to the distal portion; and at least one tensioning actuator coupled with the proximal portion and the tensioning member for applying tension to the tensioning member to deform the distal portion to allow it to conform generally to a shape of the valve annulus. In various embodiment, this steerable guide catheter may have any of the features of the catheter device described above.

In some embodiments of the system, a distal portion of the guide sheath is detachable from a proximal portion of the guide sheath to remain in attached to the valve annulus after an annulus treatment procedure. For example, the detachable distal portion may comprise a tubular member comprising Dacron or the like. In some embodiments, the detachable distal portion is cinchable to tighten the mitral valve annulus.

In some embodiments, the system may further include at least one urging member coupled with at least one of the shaped guide catheter, the steerable guide catheter and the guide sheath. For example, the urging member may comprise an expandable member for expanding within a space in a left ventricle formed by a left ventricular wall, at least one mitral valve leaflet and chordae tendiniae of the heart. Alternatively, the urging member may comprise at least one magnet coupled with at least one of the shaped guide catheter, the steerable guide catheter and the guide sheath for applying attractive magnetic force between itself and an oppositely charged magnet disposed in a coronary sinus adjacent the valve annulus.

Any suitable device or combination of devices may be advanced into contact with the mitral valve annulus in various embodiments. In some embodiments, for example, the system includes an anchor delivery device passable through the guide sheath to contact and apply coupled anchors to the mitral valve annulus. The system may additionally or alternatively include a visualization device passable through the guide sheath to facilitate visualization of the mitral valve annulus. For example, the visualization device may comprises, but is not limited to, an ultrasound device, a camera, an endoscope or a fiber optic device.

These and other aspects and embodiments are described more fully below with reference to the drawing figures.

### BRIEF DESCRIPTION OF THE DRAWINGS

FIG. 1 is a cross-sectional view of a heart with a flexible anchor delivery device being positioned for treatment of a mitral valve annulus, according to one embodiment of the present invention;

FIGS. 2A and 2B are cross-sectional views of a portion of a heart, schematically showing positioning of a flexible device for treatment of a mitral valve annulus, according to one embodiment of the present invention;

FIGS. 2C and 2D are cross-sectional views of a portion of a heart, showing positioning of a flexible anchor delivery device for treatment of a mitral valve annulus, according to one embodiment of the present invention;

FIG. 3 is a perspective view of a distal portion of an anchor delivery device, according to one embodiment of the invention;

FIG. 4. is a perspective view of a segment of a distal portion of an anchor delivery device, with anchors in an undeployed shape and position;

FIG. 5 is a different perspective view of the segment of the device shown in FIG. 4;

FIG. 6. is a perspective view of a segment of a distal portion of an anchor delivery device, with anchors in a deployed shape and position;

FIGS. 7A-7E are cross-sectional views of an anchor delivery device, illustrating a method for delivering anchors to valve annulus tissue, according to one embodiment of the invention;

FIGS. 8A and 8B are top-views of a plurality of anchors coupled to a self-deforming coupling member or “backbone,” with the backbone shown in an undeployed shape and a deployed shape;

FIGS. 9A-9C are various perspective views of a distal portion of a flexible anchor delivery device according to one embodiment of the present invention;

FIGS. 10A-10F demonstrate a method for applying anchors to a valve annulus and cinching the anchors to tighten the annulus, using an anchor delivery device according to an embodiment of the invention;

FIG. 11 shows a heart in cross-section with a guide catheter device advanced through the aorta into the left ventricle according to an embodiment of the invention;

FIGS. 12A-12F demonstrate a method for advancing an anchor delivery device to a position for treating a heart vavle according to an embodiment of the invention; and

FIGS. 13A and 13B are side cross-sectional views of a guide catheter device for facilitating positioning of an anchor delivery device according to an embodiment of the invention.

### DETAILED DESCRIPTION OF THE INVENTION

Devices, systems and methods of the present invention are generally used to facilitate transvascular, minimally invasive and other “less invasive” surgical procedures, by facilitating the delivery of treatment devices at a treatment site. Although the following description focuses on use of devices and methods of the invention for mitral valve repair, the devices and methods may be used in any suitable procedure, both cardiac and non-cardiac. When used for treatment of a cardiac valve annulus, the inventive methods generally involve contacting an anchor delivery device with a length of the valve annulus, delivering a plurality of coupled anchors from the anchor delivery device, and drawing the anchors together to tighten the annulus. Devices include an elongate catheter having a housing at or near the distal end for releasably housing a plurality of coupled anchors, as well as delivery devices for facilitating advancement and/or positioning of an anchor delivery device. Devices may be positioned such that the housing abuts or is close to valve annular tissue, such as in a location within the left ventricle defined by the left ventricular wall, a mitral valve leaflet and chordae tendineae. Self-securing anchors having any of a number of different configurations may be used in some embodiments. Additional devices include delivery devices for facilitating delivery and/or placement of an anchor delivery device at a treatment site.

In many cases, methods of the present invention will be performed on a beating heart. Access to the beating heart may be accomplished by any available technique, including intravascular, transthoracic, and the like. In addition to beating heart access, the methods of the present invention may be used for intravascular stopped heart access as well as stopped heart open chest procedures.

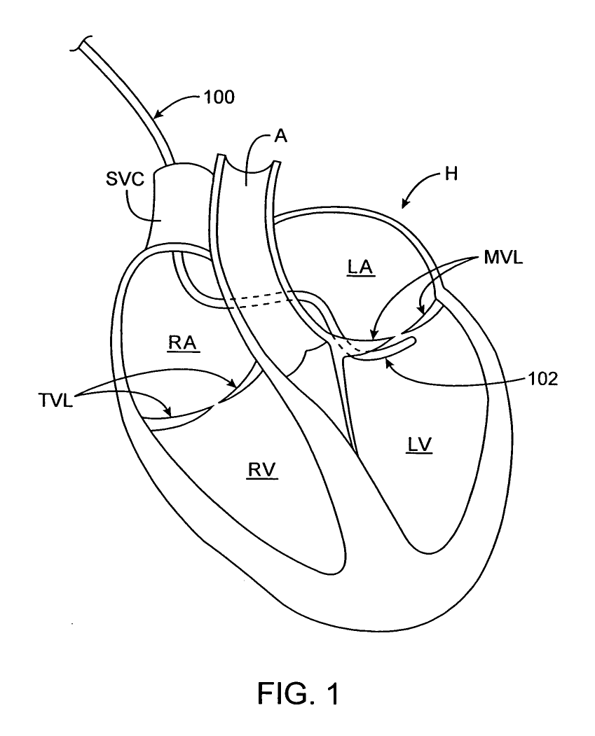

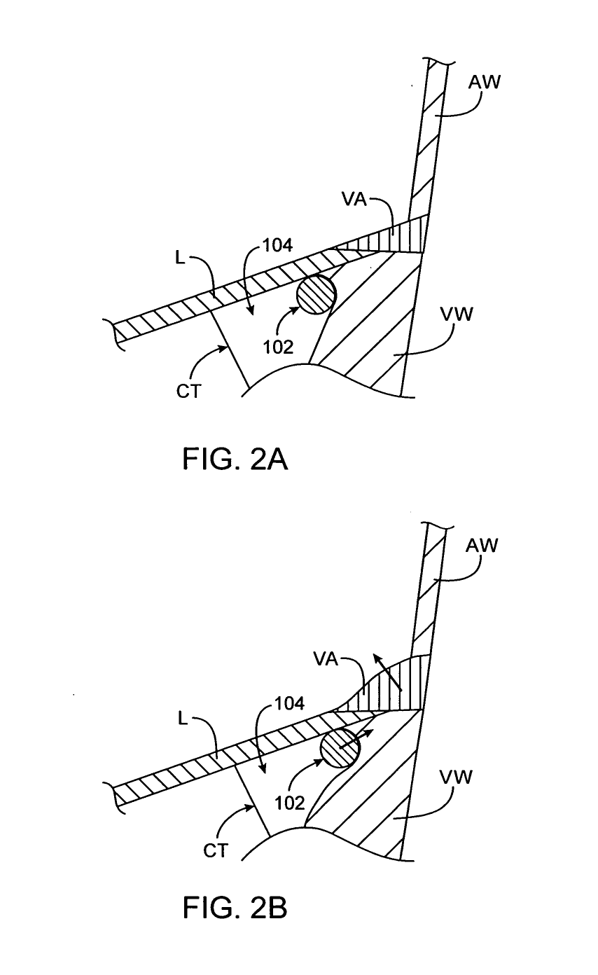

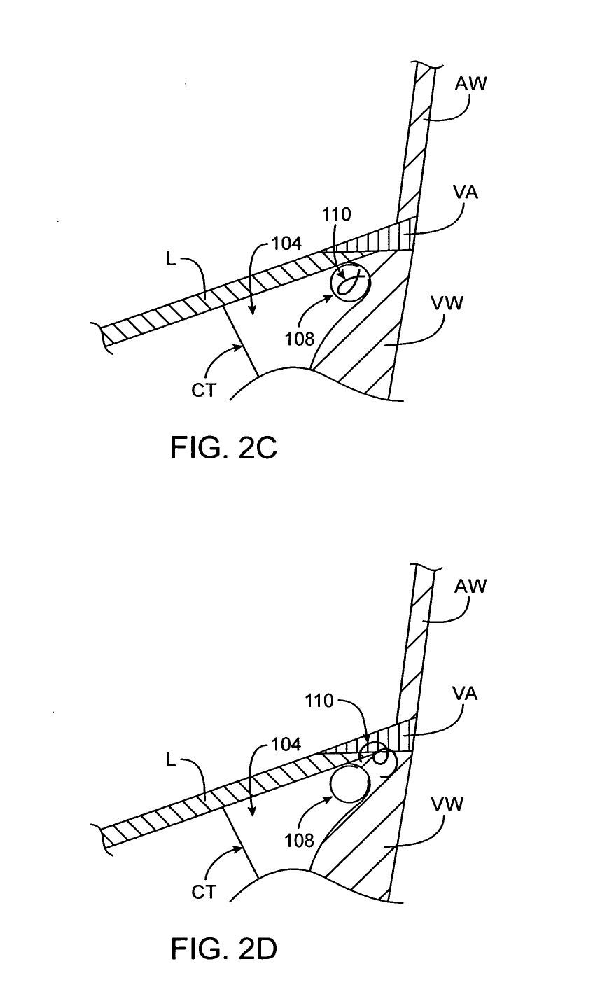

Referring now to FIG. 1, a heart H is shown in cross section, with an elongate anchor delivery device 100 introduced within the heart H. Generally, delivery device 100 comprises an elongate body with a distal portion 102 configured to deliver anchors to a heart valve annulus. (In FIGS. 1, 2A and 2B, distal portion 102 is shown diagrammatically without anchors or anchor-delivery mechanism to enhance clarity of the figures.) In some embodiments, the elongate body comprises a rigid shaft, while in other embodiments it comprises a flexible catheter, so that distal portion 102 may be positioned in the heart H and under one or more valve leaflets to engage a valve annulus via a transvascular approach. Transvascular access may be gained, for example, through the internal jugular vein (not shown) to the superior vena cava SVC to the right atrium RA, across the interatrial septum to the left atrium LA, and then under one or more mitral valve leaflets MVL to a position within the left ventricle (LV) under the valve annulus (not shown). Alternatively, access to the heart may be achieved via the femoral vein and the inferior vena cava. In other embodiments, access may be gained via the coronary sinus (not shown) and through the atrial wall into the left atrium. In still other embodiments, access may be achieved via a femoral artery and the aorta, into the left ventricle, and under the mitral valve. Any other suitable access route is also contemplated within the scope of the present invention.

In other embodiments, access to the heart H may be transthoracic, with delivery device 100 being introduced into the heart via an incision or port on the heart wall. Even open heart surgical procedures may benefit from methods and devices of the invention. Furthermore, some embodiments may be used to enhance procedures on the tricuspid valve annulus, adjacent the tricuspid valve leaflets TVL, or any other cardiac or vascular valve. Therefore, although the following description typically focuses on minimally invasive or less invasive mitral valve repair for treating mitral regurgitation, the invention is in no way limited to that use.

With reference now to FIGS. 2A and 2B, a method for positioning delivery device 100 for treating a mitral valve annulus VA is depicted diagrammatically in a cross-sectional view. First, as in FIG. 2A, distal portion 102 is positioned in a desired location under a mitral valve leaflet L and adjacent a ventricular wall VW. (Again, distal portion 102 is shown without anchors or anchor-delivery mechanism for demonstrative purposes.) The valve annulus VA generally comprises an area of heart wall tissue at the junction of the ventricular wall VW and the atrial wall AW that is relatively fibrous and, thus, significantly stronger that leaflet tissue and other heart wall tissue.

Distal portion 102 may be advanced into position under the valve annulus by any suitable technique, some of which are described below in further detail. Generally, distal portion 102 may be used to deliver anchors to the valve annulus, to stabilize and/or expose the annulus, or both. In one embodiment, using a delivery device having a flexible elongate body as shown in FIG. 1, a flexible distal portion 102 may be passed from the right atrium RA through the interatrial septum in the area of the foramen ovale (not shown—behind the aorta A), into the left atrium LA and thus the left ventricle LV. Alternatively, flexible distal portion 102 may be advanced through the aorta A and into the left ventricle LV, for example using access through a femoral artery. Oftentimes, distal portion 102 will then naturally travel, upon further advancement, under the posterior valve leaflet L into a space defined above a subvalvular space 104 roughly defined for the purposes of this application as a space bordered by the inner surface of the left ventricular wall VW, the inferior surface of mitral valve leaflets L, and cordae tendineae CT connected to the ventricular wall VW and the leaflet L. It has been found that a flexible anchor delivery catheter, such as the delivery devices of the present invention, when passed under the mitral valve via an intravascular approach, often enters subvalvular space 104 relatively easily and may be advanced along space 104 either partially or completely around the circumference of the valve. Once in space 104, distal portion 102 may be conveniently positioned at the intersection of the valve leaflet(s) and the ventricular wall VW, which intersection is immediately adjacent or very near to the valve annulus VA, as shown in FIG. 2A. These are but examples of possible access routes of an anchor delivery device to a valve annulus, and any other access routes may be used.

In some embodiments, distal portion 102 includes a shape-changing portion which enables distal portion 102 to conform to the shape of the valve annulus VA. The catheter may be introduced through the vasculature with the shape-changing distal portion in a generally straight, flexible configuration. Once it is in place beneath the leaflet at the intersection between the leaflet and the interior ventricular wall, the shape of distal portion 102 is changed to conform to the annulus and usually the shape is “locked” to provide sufficient stiffness or rigidity to permit the application of force from distal portion 102 to the annulus. Shaping and optionally locking distal portion 102 may be accomplished in any of a number of ways. For example, in some embodiments, a shape-changing portion may be sectioned, notched, slotted or segmented and one of more tensioning members such as tensioning cords, wires or other tensioning devices coupled with the shape-changing portion may be used to shape and rigidify distal portion 102. A segmented distal portion, for example, may include multiple segments coupled with two tensioning members, each providing a different direction of articulation to the distal portion. A first bend may be created by tensioning a first member to give the distal portion a C-shape or similar shape to conform to the valve annulus, while a second bend may be created by tensioning a second member to articulate the C-shaped member upwards against the annulus. In another embodiment, a shaped expandable member, such as a balloon, may be coupled with distal portion 102 to provide for shape changing/deforming. In various embodiments, any configurations and combinations may be used to give distal portion 102 a desired shape.

In transthoracic and other embodiments, distal portion 102 may be pre-shaped, and the method may simply involve introducing distal portion 102 under the valve leaflets. The pre-shaped distal portion 102 may be rigid or formed from any suitable super-elastic or shape memory material, such as nitinol, spring stainless steel, or the like.

In addition to delivering anchors to the valve annulus VA, delivery device 100 (and specifically distal portion 102) may be used to stabilize and/or expose the valve annulus VA. Such stabilization and exposure are described fully in U.S. patent application Ser. No. 10/656797, which was previously incorporated by reference. For example, once distal portion 102 is positioned under the annulus, force may be applied to distal portion 102 to stabilize the valve annulus VA, as shown in FIG. 2B. Such force may be directed in any suitable direction to expose, position and/or stabilize the annulus. For example, upward and lateral force is shown in FIG. 2B by the solid-headed arrow drawn from the center of distal portion 102. In other cases, only upward, only lateral, or any other suitable force(s) may be applied. With application of force to distal portion 102, the valve annulus VA is caused to rise or project outwardly, thus exposing the annulus for easier viewing and access. The applied force may also stabilize the valve annulus VA, also facilitating surgical procedures and visualization.

Some embodiments may include a stabilization component as well as an anchor delivery component. For example, some embodiments may include two flexible members, one for contacting the atrial side of a valve annulus and the other for contacting the ventricular side. In some embodiments, such flexible members may be used to “clamp” the annulus between them. One of such members may be an anchor delivery member and the other may be a stabilization member, for example. Any combination and configuration of stabilization and/or anchor delivery members is contemplated.

Referring now to FIGS. 2C and 2D, an anchor delivery device 108 is shown delivering an anchor 110 to a valve annulus VA. Of course, these are again representational figures and are not drawn to scale. Anchor 110 is shown first housed within delivery device 108 (FIG. 2C) and then delivered to the annulus VA (FIG. 2D). As is shown, in one embodiment anchors 110 may have a relatively straight configuration when housed in delivery device 108, perhaps with two sharpened tips and a loop in between the tips. Upon deployment from delivery device 108, the tips of anchor 110 may curve in opposite directions to form two semi-circles, circles, ovals, overlapping helices or the like. This is but one example of a type of self-securing anchor which may be delivered to a valve annulus. Typically, multiple coupled anchors 110 are delivered, and the anchors 110 are drawn together to tighten the valve annulus. Methods for anchor delivery and for drawing anchors together are described further below.

Although delivery device 108 is shown having a circular cross-sectional shape in FIGS. 2C and 2D, it may alternatively have any other suitable shape. In one embodiment, for example, it may be advantageous to provide a delivery device having an ovoid or elliptical cross-sectional shape. Such a shape may help ensure that the device is aligned, when positioned between in a corner formed by a ventricular wall and a valve leaflet, such that one or more openings in the delivery device is oriented to deliver the anchors into valve annulus tissue. To further enhance contacting of the valve annulus and/or orientation of the delivery device, some embodiments may further include an expandable member, coupled with the delivery device, which expands to urge or press or wedge the delivery device into the corner formed by the ventricle wall and the leaflet to contact the valve annulus. Such enhancements are described further below.

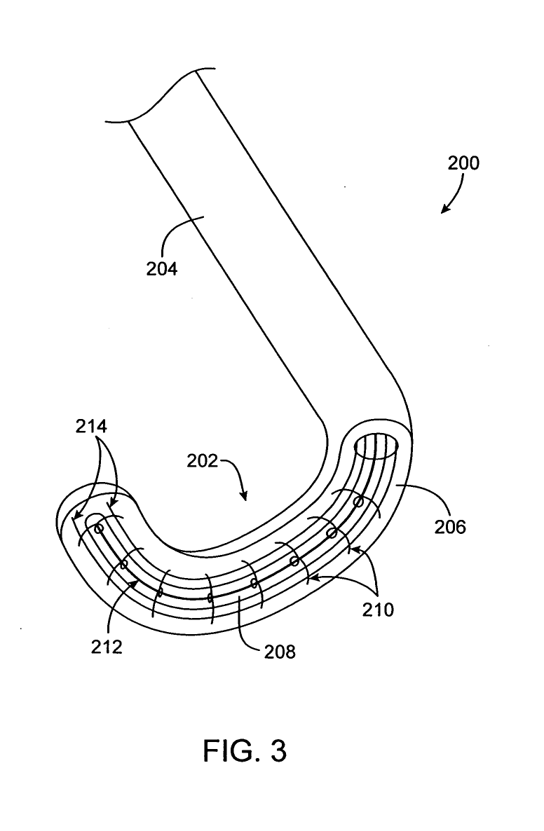

With reference now to FIG. 3, one embodiment of a portion of an anchor delivery device 200 suitably includes an elongate shaft 204 having a distal portion 202 configured to deliver a plurality of anchors 210, coupled with a tether 212, to tissue of a valve annulus. Tethered anchors 210 are housed within a housing 206 of distal portion 202, along with one or more anchor retaining mandrels 214 and an expandable member 208. Many variations may be made to one or more of these features, and various parts may be added or eliminated, without departing from the scope of the invention. Some of these variations are described further below, but no specific embodiment(s) should be construed to limit the scope of the invention as defined by the appended claims.

Housing 206 may be flexible or rigid in various embodiments. In some embodiments, for example, flexible housing 206 may be comprised of multiple segments configured such that housing 206 is deformable by tensioning a tensioning member coupled to the segments. In some embodiments, housing 206 is formed from an elastic material having a geometry selected to engage and optionally shape or constrict the valve annulus. For example, the rings may be formed from super-elastic material, shape memory alloy such as Nitinol, spring stainless steel, or the like. In other instances, housing 206 could be formed from an inflatable or other structure can be selectively rigidified in situ, such as a gooseneck or lockable element shaft, any of the rigidifying structures described above, or any other rigidifying structure.

“Anchors,” for the purposes of this application, is defined to mean any fasteners. Thus, anchors 210 may comprise C-shaped or semicircular hooks, curved hooks of other shapes, straight hooks, barbed hooks, clips of any kind, T-tags, or any other suitable fastener(s). In one embodiment, as described above, anchors may comprise two tips that curve in opposite directions upon deployment, forming two intersecting semi-circles, circles, ovals, helices or the like. In some embodiments, anchors 210 are self-deforming. By “self-deforming” it is meant that anchors 210 change from a first undeployed shape to a second deployed shape upon release of anchors 210 from restraint in housing 206. Such self-deforming anchors 210 may change shape as they are released from housing 206 and enter valve annulus tissue, to secure themselves to the tissue. Thus, a crimping device or other similar mechanism is not required on distal end 202 to apply force to anchors 210 to attach them to annular tissue. Self-deforming anchors 210 may be made of any suitable material, such as a super-elastic or shape-memory material like Nitinol or spring stainless steel. In other embodiments, anchors 210 may be made of a non-shape-memory material and made be loaded into housing 206 in such a way that they change shape upon release. Alternatively, anchors 210 that are not self-deforming may be used, and such anchors may be secured to tissue via crimping, firing or the like. Even self-securing anchors may be crimped in some embodiments, to provide enhanced attachment to tissue. Delivery of anchors may be accomplished by any suitable device and technique, such as by simply releasing the anchors by hydraulic balloon delivery as discussed further below. Any number, size and shape of anchors 210 may be included in housing 206.

In one embodiment, anchors 210 are generally C-shaped or semicircular in their undeployed form, with the ends of the C being sharpened to penetrate tissue. Midway along the C-shaped anchor 210, an eyelet may be formed for allowing slidable passage of tether 212. To maintain anchors 210 in their C-shaped, undeployed state, anchors 210 may be retained within housing 206 by two mandrels 214, one mandrel 214 retaining each of the two arms of the C-shape of each anchor 210. Mandrels 214 may be retractable within elongate catheter body 204 to release anchors 210 and allow them to change from their undeployed C-shape to a deployed shape. The deployed shape, for example, may approximate a complete circle or a circle with overlapping ends, the latter appearing similar to a key ring. Such anchors are described further below, but generally may be advantageous in their ability to secure themselves to annular tissue by changing from their undeployed to their deployed shape. In some embodiments, anchors 210 are also configured to lie flush with a tissue surface after being deployed. By “flush” it is meant that no significant amount of an anchor protrudes from the surface, although some small portion may protrude.

Tether 212 may be one long piece of material or two or more pieces and may comprise any suitable material, such as suture, suture-like material, a Dacron strip or the like. Retaining mandrels 214 may also have any suitable configuration and be made of any suitable material, such as stainless steel, titanium, Nitinol, or the like. Various embodiments may have one mandrel, two mandrels, or more than two mandrels.

In some embodiments, anchors 210 may be released from mandrels 214 to contact and secure themselves to annular tissue without any further force applied by delivery device 200. Some embodiments, however, may also include one or more expandable members 208, which may be expanded to help drive anchors 210 into tissue. Expandable member(s) 208 may have any suitable size and configuration and may be made of any suitable material(s). Hydraulic systems such as expandable members are known in the art, and any known or as yet undiscovered expandable member may be included in housing 206 as part of the present invention.

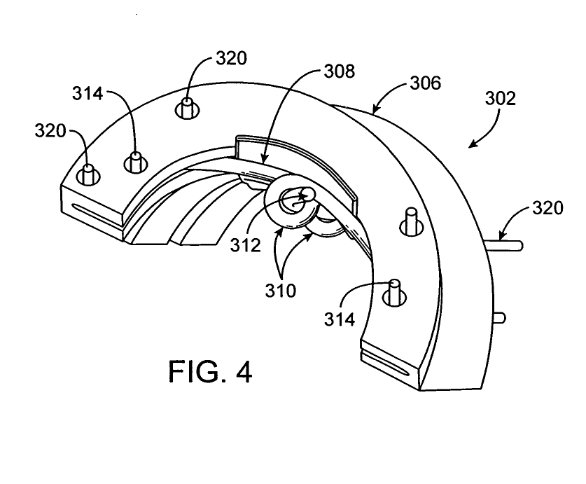

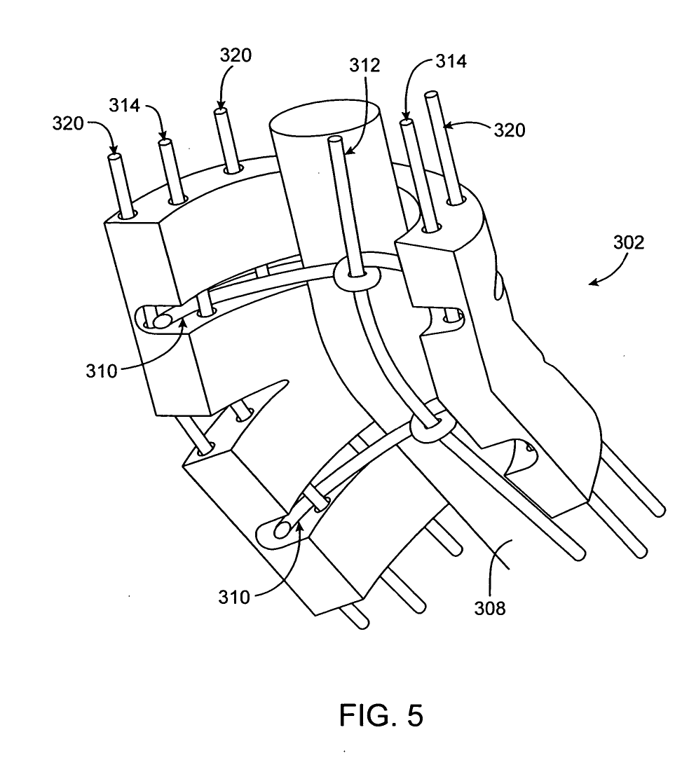

Referring now to FIGS. 4 and 5, a segment of a distal portion 302 of an anchor delivery device suitably includes a housing 306, multiple tensioning members 320 for applying tension to housing 306 to change its shape, two anchor retaining mandrels 314 slidably disposed in housing 306, multiple anchors 310 slidably coupled with a tether 312, and an expandable member 308 disposed between anchors 310 and housing 306. As can be seen in FIGS. 4 and 5, housing 306 may include multiple segments to allow the overall shape of housing 306 to be changed by applying tension to tensioning members 320. As also is evident from the drawings, “C-shaped” anchors 310 may actually have an almost straight configuration when retained by mandrels 314 in housing 306. Thus, for the purposes of this application, “C-shaped” or “semicircular” refers to a very broad range of shapes including a portion of a circle, a slightly curved line, a slightly curved line with an eyelet at one point along the line, and the like.

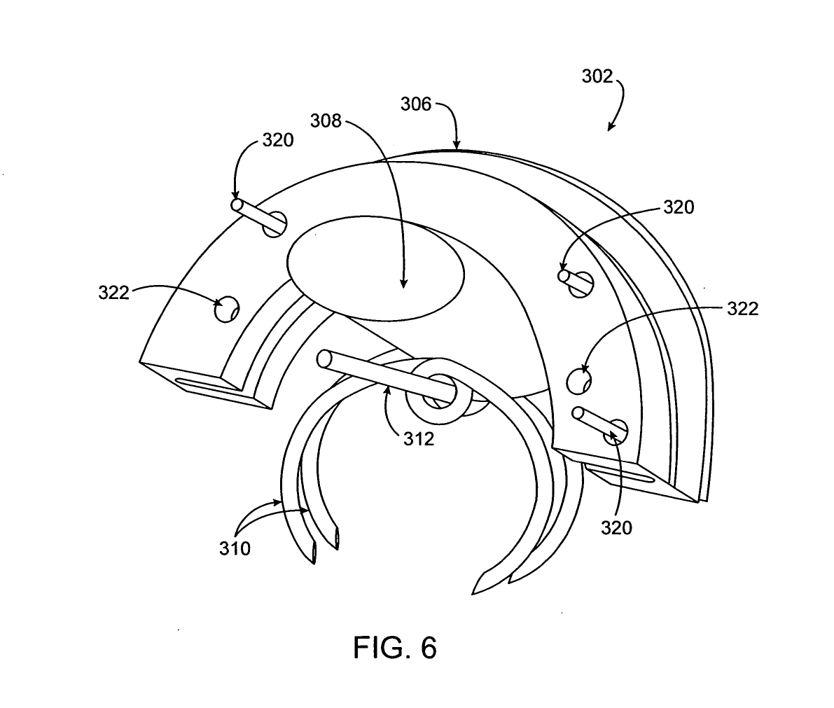

With reference now to FIG. 6, the same segment of distal portion 302 is shown, but mandrels 314 have been withdrawn from two mandrel apertures 322, to release anchors 310 from housing 306. Additionally, expandable member 308 has been expanded to drive anchors out of housing 306. Anchors 310, having been released from mandrels 314, have begun to change from their undeployed, retained shape to their deployed, released shape.

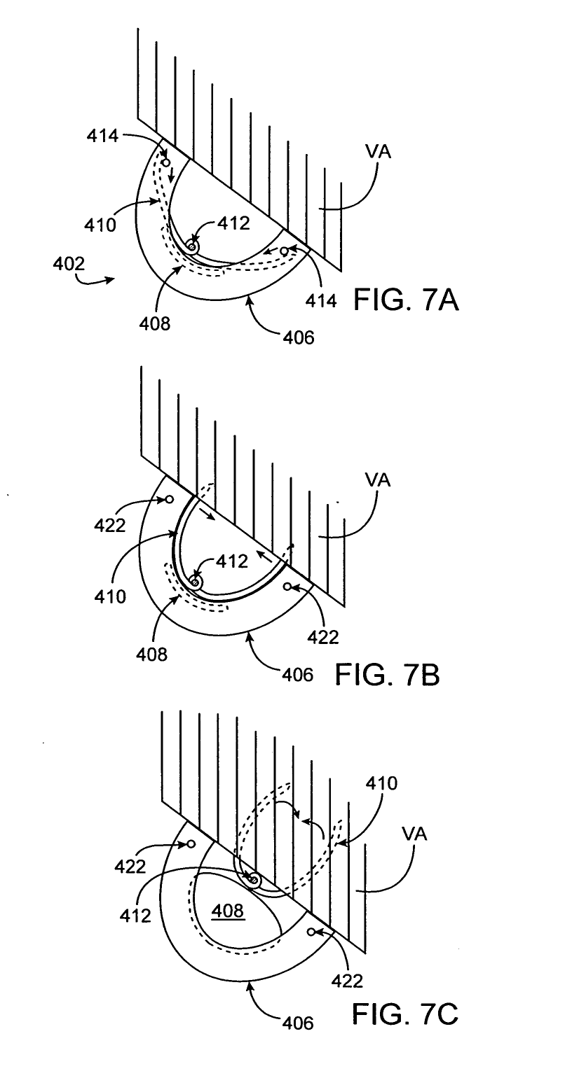

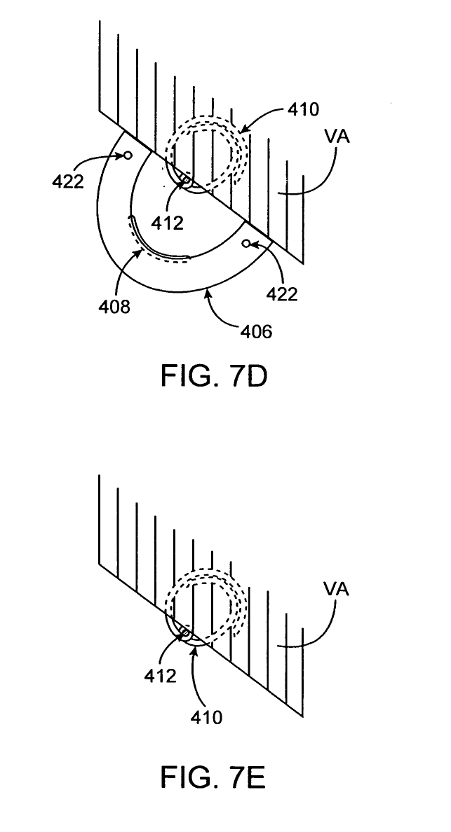

Referring now to FIGS. 7A-7E, a cross-section of a distal portion 402 of an anchor delivery device is shown in various stages of delivering an anchor to tissue of a valve annulus VA. In FIG. 7A, distal portion 402 is positioned against the valve annulus, an anchor 410 is retained by two mandrels 414, a tether 412 is slidably disposed through an eyelet on anchor 410, and an expandable member 408 is coupled with housing 406 in a position to drive anchor 410 out of housing 406. When retained by mandrels 414, anchor 410 is in its undeployed shape. As discussed above, mandrels 414 may be slidably retracted, as designated by the solid-tipped arrows in FIG. 7A, to release anchor 410. In various embodiments, anchors 410 may be released one at a time, such as by retracting mandrels 414 slowly, may be released in groups, or may all be released simultaneously, such as by rapid retraction of mandrels 414.

In FIG. 7B, anchor 410 has begun to change from its undeployed shape to its deployed shape (as demonstrated by the hollow-tipped arrows) and has also begun to penetrate the annular tissue VA. Empty mandrel apertures 422 demonstrate that mandrels 414 have been retracted at least far enough to release anchor 410. In FIG. 7B, expandable member 408 has been expanded to drive anchor 410 partially out of housing 406 and further into the valve annulus VA. Anchor 410 also continues to move from its undeployed towards its deployed shape, as shown by the hollow-tipped arrows. In FIG. 7D, anchor 410 has reached its deployed shape, which is roughly a completed circle with overlapping ends or a “key ring” shape. In FIG. 7E, delivery device 402 has been removed, leaving a tethered anchor in place in the valve annulus. Of course, there will typically be a plurality of tethered anchors secured to the annular tissue. Tether 412 may then be cinched to apply force to anchors 410 and cinch and tighten the valve annulus.

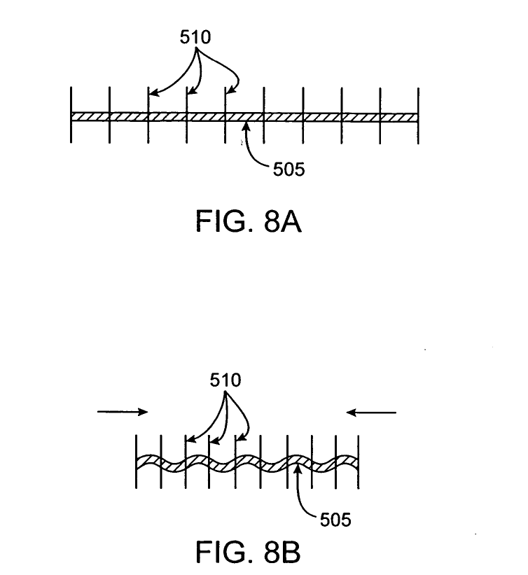

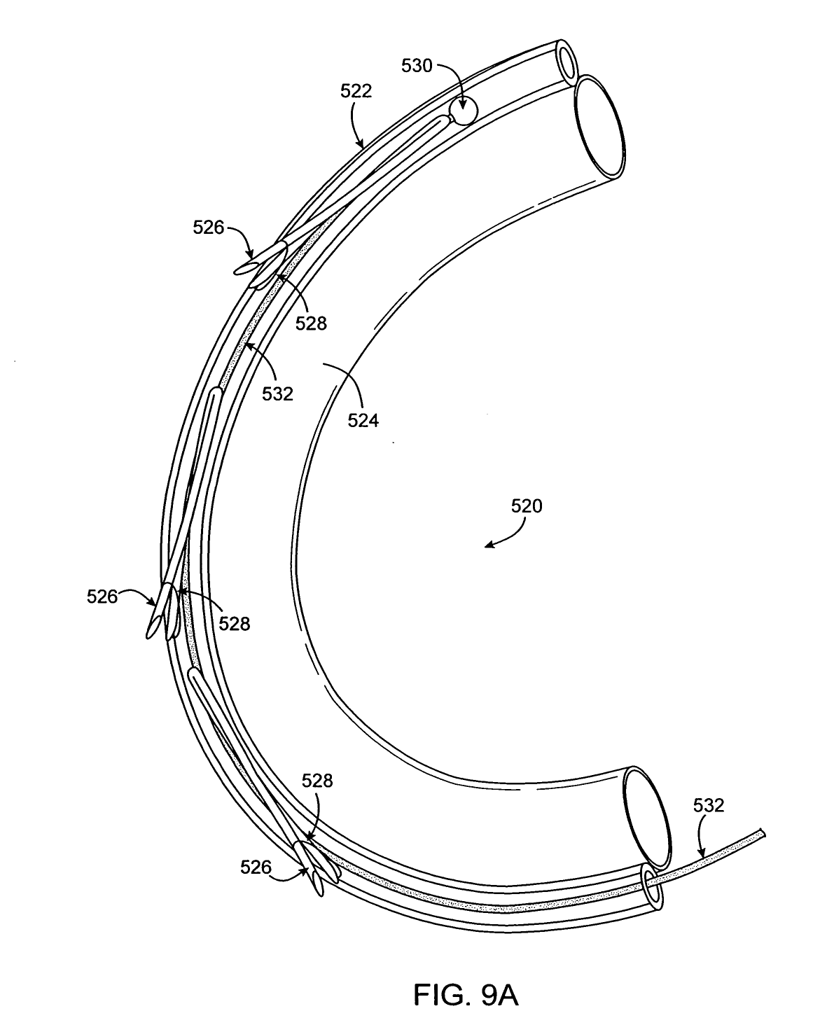

With reference now to FIGS. 8A and 8B, a diagrammatic representation of another embodiment of coupled anchors is shown. Here, anchors 510 are coupled to a self-deforming or deformable coupling member or backbone 505. Backbone 505 may be fabricated, for example, from Nitinol, spring stainless steel, or the like, and may have any suitable size or configuration. In one embodiment, as in FIG. 8A, backbone 505 is shaped as a generally straight line when held in an undeployed state, such as when restrained within a housing of an anchor deliver device. When released from the delivery device, backbone 505 may change to a deployed shape having multiple bends, as shown in FIG. 8B. By bending, backbone 505 shortens the longitudinal distance between anchors, as demonstrated by the solid-tipped arrows in FIG. 8B. This shortening process may act to cinch a valve annulus into which anchors 510 have be secured. Thus, anchors 510 coupled to backbone 505 may be used to cinch a valve annulus without using a tether or applying tethering force. Alternatively, a tether may also be coupled with anchors 510 to further cinch the annulus. In such an embodiment, backbone 505 will be at least partially conformable or cinchable, such that when force is applied to anchors 510 and backbone 505 via a tether, backbone 505 bends further to allow further cinching of the annulus.

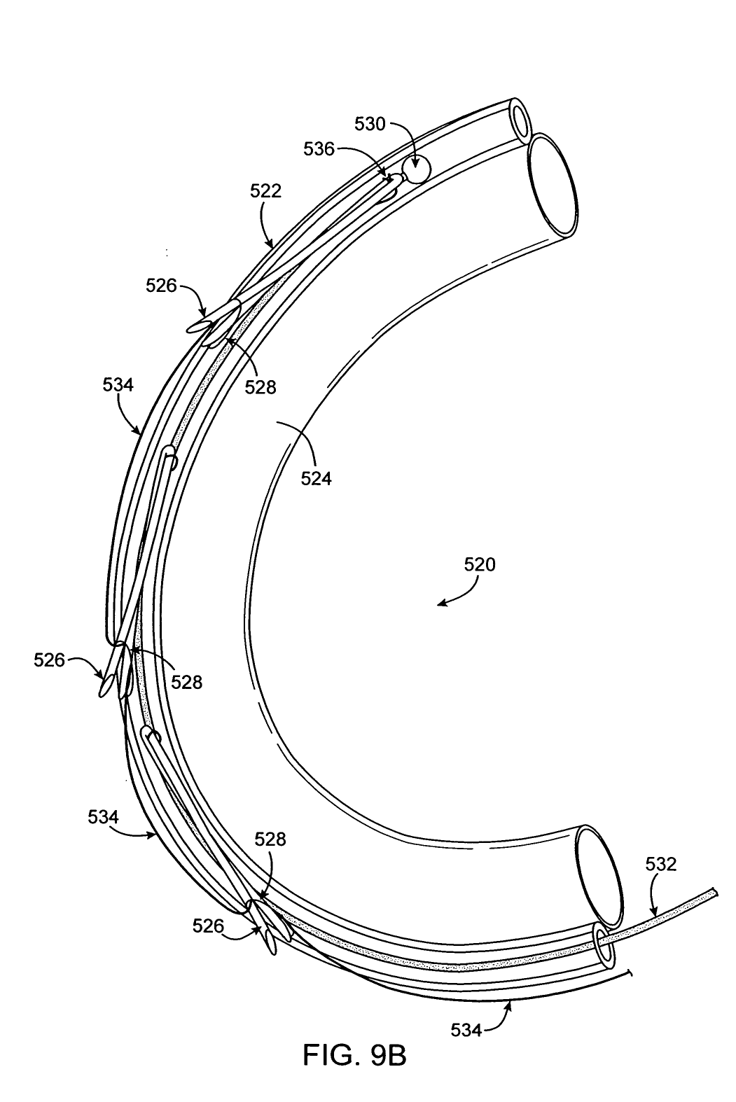

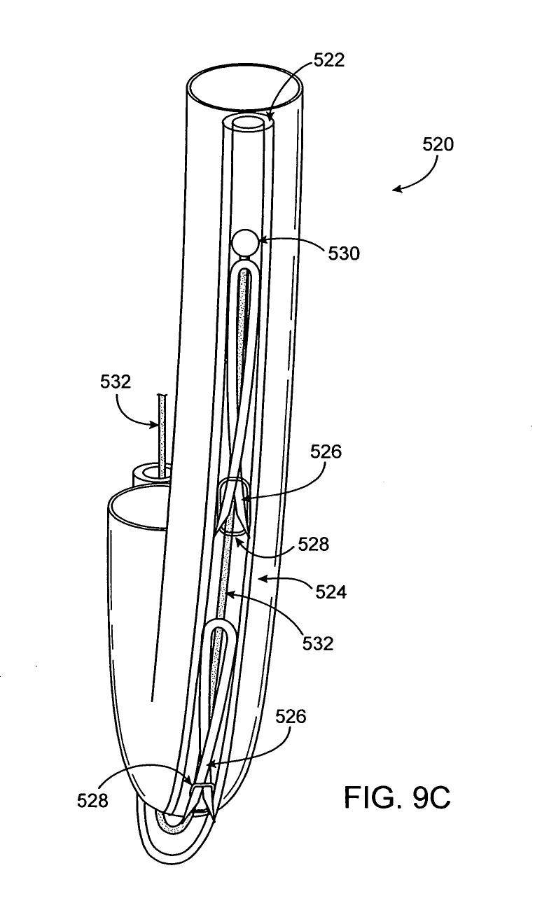

Referring now to FIGS. 9A-9C, in one embodiment a flexible distal portion of an anchor delivery device 520 suitably includes a housing 522 coupled with an expandable member 524. Housing 522 may be configured to house multiple coupled anchors 526 and an anchor contacting member 530 coupled with a pull cord 532. Housing 522 may also include multiple apertures 528 for allowing egress of anchors 526. For clarity, delivery device 520 is shown without a tether in FIGS. 9A and 9C, but FIG. 9B shows that a tether 534 may extend through an eyelet, loop or other portion of each anchor 526, and may exit each aperture 528 to allow for release of the plurality of anchors 526. The various features of this embodiment are described further below.

In the embodiment shown in FIGS. 9A-9C, anchors 526 are relatively straight and lie relatively in parallel with the long axis of delivery device 522. Anchor contacting member 530, which may comprise any suitable device, such as a ball, plate, hook, knot, plunger, piston, or the like, generally has an outer diameter that is nearly equal to or slightly less than the inner diameter of housing 522. Contacting member 530 is disposed within the housing, distal to a distal-most anchor 526, and is retracted relative to housing 522 by pulling pull cord 532. When retracted, anchor contacting member 530 contacts and applies force to a distal-most anchor 526 to release cause that anchor 526 to exit housing 522 via one of the apertures 528. Contacting member 530 is then pulled farther proximally to contact and apply force to the next anchor 526 to deploy that anchor 526, and so on.

Retracting contacting member 530 to push anchors 526 out of apertures 528 may help cause anchors 526 to avidly secure themselves to adjacent tissue. Using anchors 526 that are relatively straight/flat when undeployed allows anchors 526 with relatively large deployed sizes to be disposed in (and delivered from) a relatively small housing 522. In one embodiment, for example, anchors 526 that deploy into a shape approximating two intersecting semi-circles, circles, ovals, helices, or the like, and that have a radius of one of the semi-circles of about 3 mm may be disposed within a housing 522 having a diameter of about 5 French (1.67 mm) and more preferably 4 French (1.35 mm) or even smaller. Such anchors 526 may measure about 6 mm or more in their widest dimension. These are only examples, however, and other larger or smaller anchors 526 may be disposed within a larger or smaller housing 522. Furthermore, any convenient number of anchors 526 may be disposed within housing 522. In one embodiment, for example, housing 522 may hold about 1-20 anchors 526, and more preferably about 3-10 anchors 526. Other embodiments may hold more anchors 526.

Anchor contacting member 530 and pull cord 532 may have any suitable configuration and may be manufactured from any material or combination of materials. In alternative embodiments, contacting member 530 may be pushed by a pusher member to contact and deploy anchors 526. Alternatively, any of the anchor deployment devices and methods previously described may be used.

Tether 534, as shown in FIG. 9B, may comprise any of the tethers 534 or tether-like devices already described above, or any other suitable device. Tether 534 is generally attached to a distal-most anchor 526 at an attachment point 536. The attachment itself may be achieved via a knot, weld, adhesive, or by any other suitable attachment means. Tether 234 then extends through an eyelet, loop or other similar configuration on each on each of the anchors 526 so as to be slidably coupled with the anchors 526. In the embodiment shown, tether 534 exits each aperture 528, then enters the next-most-proximal aperture, passes slidably through a loop on an anchor 526, and exits the same aperture 528. By entering and exiting each aperture 528, tether 534 allows the plurality of anchors 526 to be deployed into tissue and cinched. Other configurations of housing 522, anchors 526 and tether 534 may alternatively be used. For example, housing 522 may include a longitudinal slit through which tether 534 may pass, thus allowing tether 534 to reside wholly within housing before deployment.

Expandable member 524 is an optional feature of anchor delivery device 520, and thus may be included in some embodiments and not in others. In other words, a distal portion of anchor delivery device 520 may include housing, contents of housing, and other features either with or without an attached expandable member. Expandable member 524 may comprise any suitable expandable member currently known or discovered in the future, and any method and substance(s) may be used to expand expandable member 524. Typically, expandable member 524 will be coupled with a surface of housing 522, will have a larger radius than housing 522, and will be configured such that when it is expanded as housing 522 nears or contacts the valve annulus, expandable member 524 will push or press housing 522 into enhanced contact with the annulus. For example, expandable member 524 may be configured to expand within a space near the corner formed by a left ventricular wall and a mitral valve leaflet.

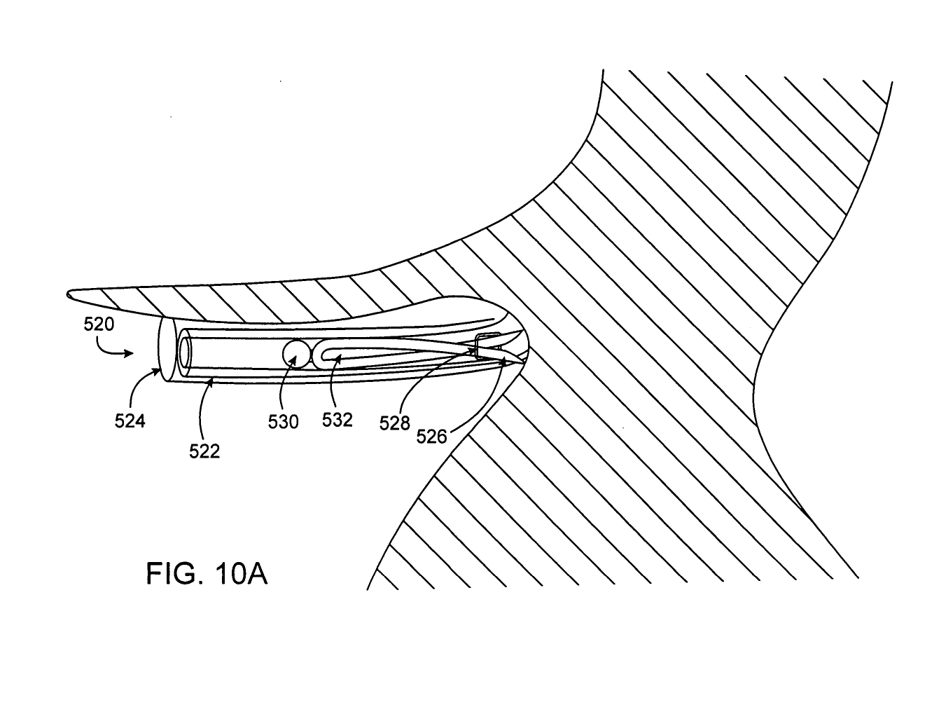

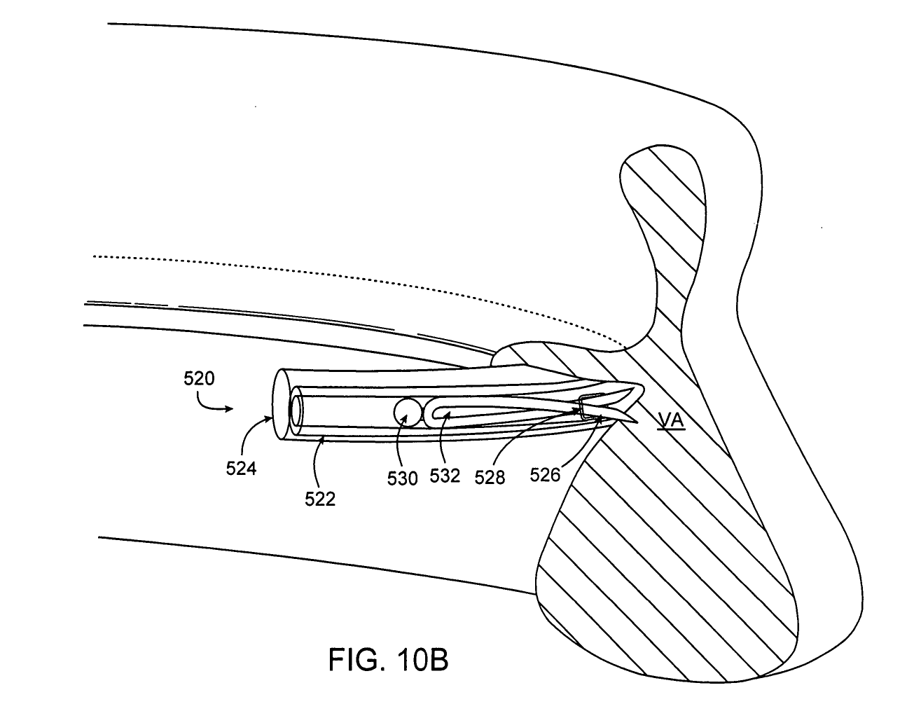

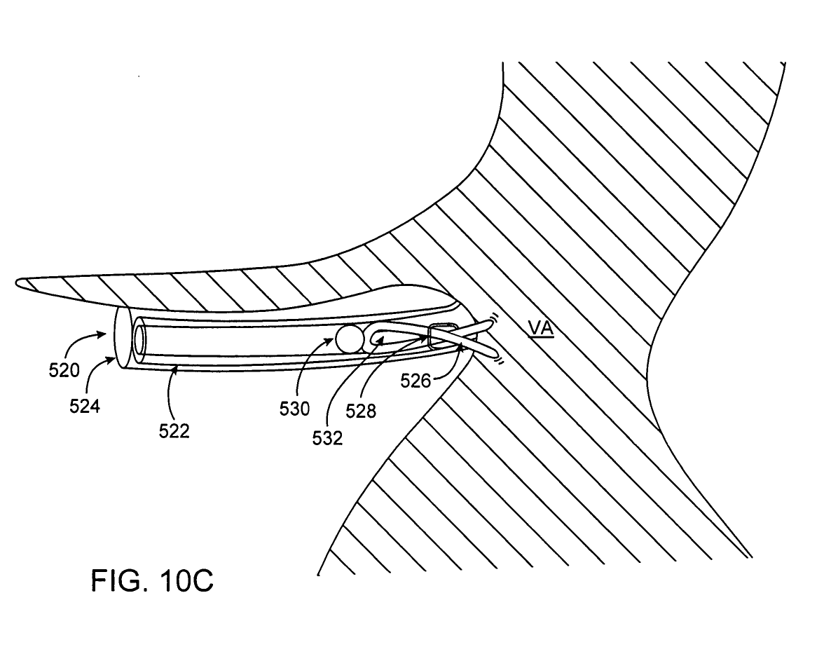

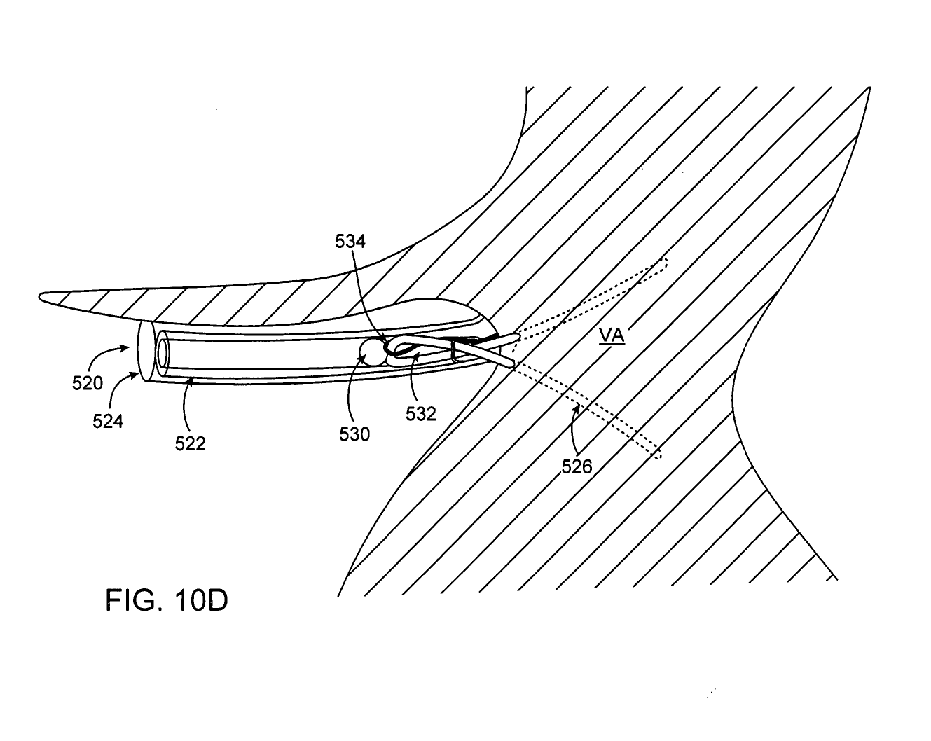

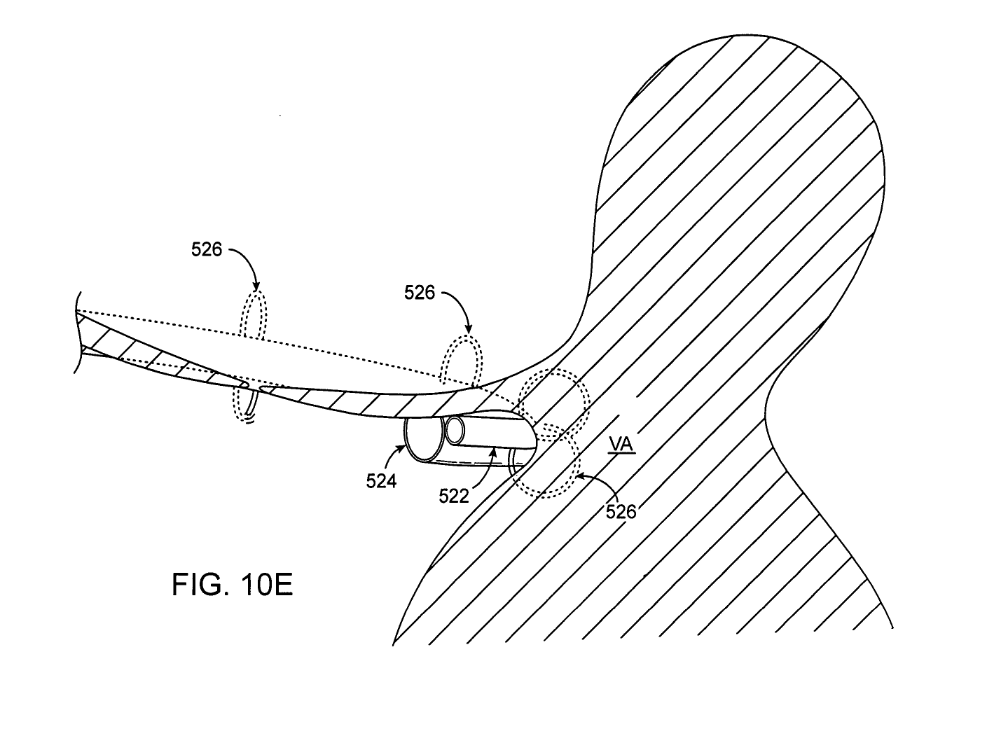

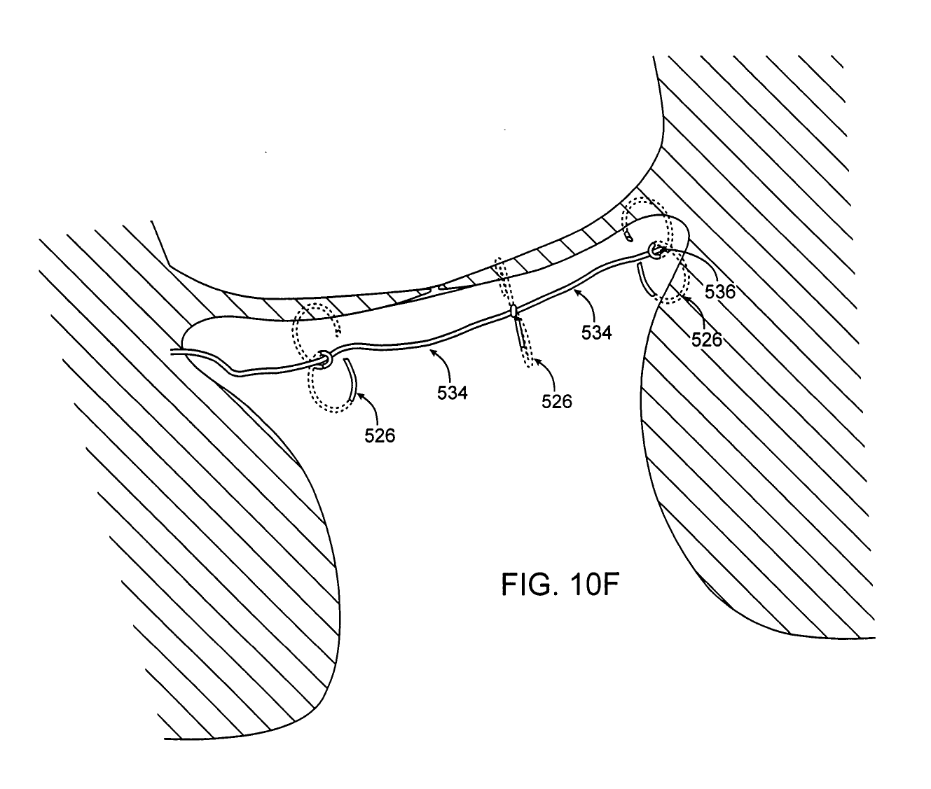

With reference now to FIGS. 10A-10F, a method is shown for applying a plurality of tethered anchors 526 to a valve annulus VA in a heart. As shown in FIG. 10A, an anchor delivery device 520 is first contacted with the valve annulus VA such that openings 528 are oriented to deploy anchors 526 into the annulus. Such orientation may be achieved by any suitable technique. In one embodiment, for example, a housing 522 having an elliptical cross-sectional shape may be used to orient openings 528. As just described, contact between housing 522 and the valve annulus VA may be enhanced by expanding expandable member 524 to wedge housing within a corner adjacent the annulus.

Generally, delivery device 520 may be advanced into any suitable location for treating any valve by any suitable advancing or device placement method. Many catheter-based, minimally invasive devices and methods for performing intravascular procedures, for example, are well known, and any such devices and methods, as well as any other devices or method later developed, may be used to advance or position delivery device 520 in a desired location. For example, in one embodiment a steerable guide catheter is first advanced in retrograde fashion through an aorta, typically via access from a femoral artery. The steerable catheter is passed into the left ventricle of the heart and thus into the space formed by the mitral valve leaflets, the left ventricular wall and cordae tendineae of the left ventricle. Once in this space, the steerable catheter is easily advanced along a portion (or all) of the circumference of the mitral valve. A sheath is advanced over the steerable catheter within the space below the valve leaflets, and the steerable catheter is removed through the sheath. Anchor delivery device 520 may then be advanced through the sheath to a desired position within the space, and the sheath may be removed. In some cases, an expandable member coupled to delivery device 520 may be expanded to wedge or otherwise move delivery device 520 into the corner formed by the left ventricular wall and the valve leaflets to enhance its contact with the valve annulus. Of course, this is but one exemplary method for advancing delivery device 520 to a position for treating a valve, and any other suitable method, combination of devices, etc. may be used.

As shown in FIG. 10B, when delivery device 520 is positioned in a desired location for deploying anchors 526, anchor contacting member 530 is retracted to contact and apply force to a most-distal anchor 526 to begin deploying anchor 526 through aperture 528 and into tissue of the valve annulus VA. FIG. 10C show anchor 526 further deployed out of aperture 528 and into valve annulus VA. FIG. 10D shows the valve annulus VA transparently so that further deployment of anchors 526 can be seen. As shown, in one embodiment of the invention, anchors 526 include two sharpened tips that move in opposite directions upon release from housing 522 and upon contacting the valve annulus VA. Between the two sharpened tips, an anchor 526 may be looped or have any other suitable eyelet or other device for allowing slidable coupling with a tether 534.

Referring now to FIG. 10E, anchors 526 are seen in their fully deployed or nearly fully deployed shape, with each pointed tip (or “arm”) of each anchor 526 having curved to form a circle or semi-circle. Of course, in various embodiments anchors 526 may have any other suitable deployed and undeployed shapes, as described more fully above. FIG. 10F shows anchors 526 deployed into the valve annulus VA and coupled with tether 534, with the distal-most anchor 526 coupled attached fixedly to tether 524 at attachment point 536. At this stage, tether 534 may be cinched to tighten the annulus, thus reducing valve regurgitation. In some embodiments, valve finction may be monitored by means such as echocardiogram and/or fluoroscopy, and tether 534 may be cinched, loosened, and adjusted to achieve a desired amount of tightening as evident via the employed visualization technique(s). When a desired amount of tightening is achieved, tether 534 is then attached to a most-proximal anchor 526 (or two or more most-proximal anchors 526), using any suitable technique, and tether 534 is then cut proximal to the most-proximal anchor 526, thus leaving the cinched, tethered anchors 526 in place along the valve annulus VA. Attachment of tether 534 to the most-proximal anchor(s) 526 may be achieved via adhesive, knotting, crimping, tying or any other technique, and cutting tether 534 may also be performed via any technique, such as with a cutting member coupled with housing 522.

In one embodiment, cinching tether 534, attaching tether 534 to most-proximal anchor 526, and cutting tether 534 are achieved using a termination device (not shown). The termination device may comprise, for example, a catheter advancable over tether 534 that includes a cutting member and a nitinol knot or other attachment member for attaching tether 534 to most-proximal anchor. The termination catheter may be advanced over tether 534 to a location at or near the proximal end of the tethered anchors 526. It may then be used to apply opposing force to the most-proximal anchor 526 while tether 534 is cinched. Attachment and cutting members may then be used to attach tether 534 to most-proximal anchor 526 and cut tether 534 just proximal to most-proximal anchor 526. Such a termination device is only one possible way of accomplishing the cinching, attachment and cutting steps, and any other suitable device(s) or technique(s) may be used.

In some embodiments, it may be advantageous to deploy a first number of anchors 526 along a first portion of a valve annulus VA, cinch the first anchors to tighten that portion of the annulus, move the delivery device 520 to another portion of the annulus, and deploy and cinch a second number of anchors 526 along a second portion of the annulus. Such a method may be more convenient, in some cases, than extending delivery device 520 around all or most of the circumference of the annulus, and may allow a shorter, more maneuverable housing 522 to be used.

Referring now to FIG. 11, a cross-sectional depiction of a heart H is shown with an anchor delivery device guide catheter 550 advanced through the aorta A and into the left ventricle LV. Guide catheter 550 is generally a flexible elongate catheter which may have one or more curves or bends toward its distal end to facilitate placement of the distal end of catheter 550 in a subannular space 552. Subannular space 552, which has been described above in detail, is generally defined by the left ventricular wall, the mitral valve leaflets MVL, and cordae tendiniae, and travels along most or all of the circumference of the valve annulus. The distal end of guide catheter 550 may be configured to be positioned at an opening into space 552 or within space 552, such that subsequent catheter devices may be passed through guide catheter 550 into space 552.

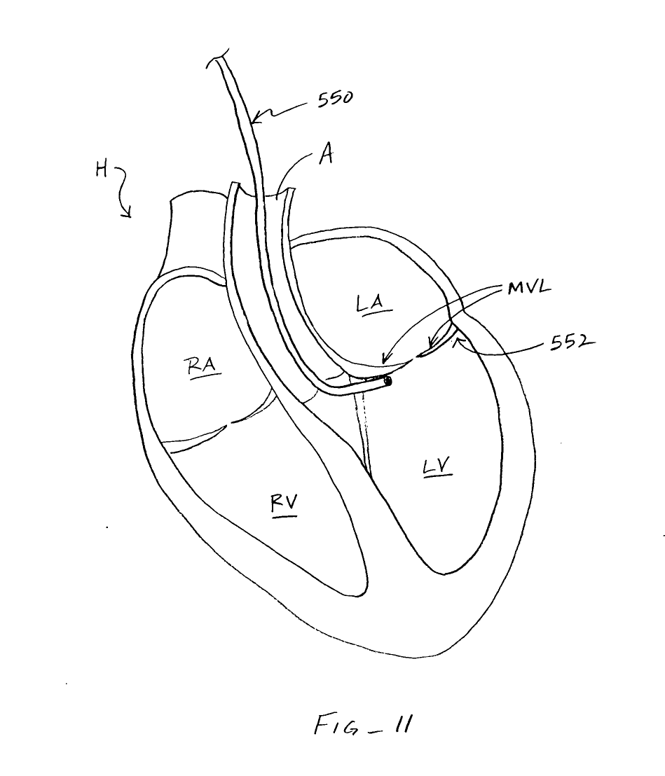

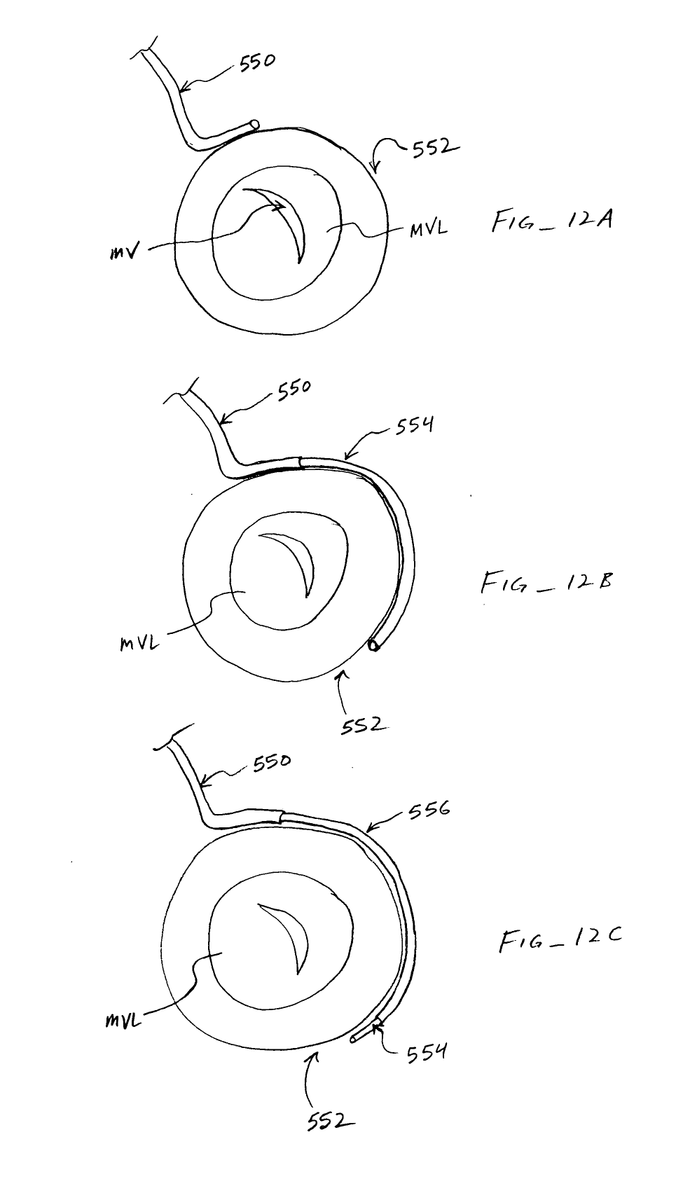

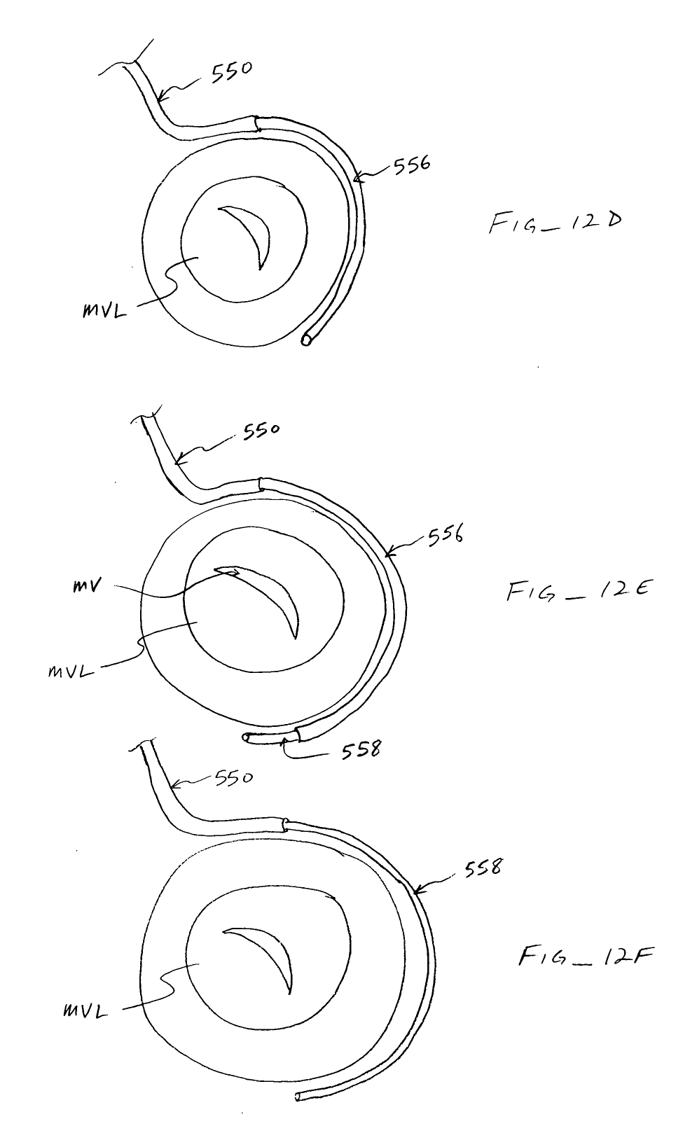

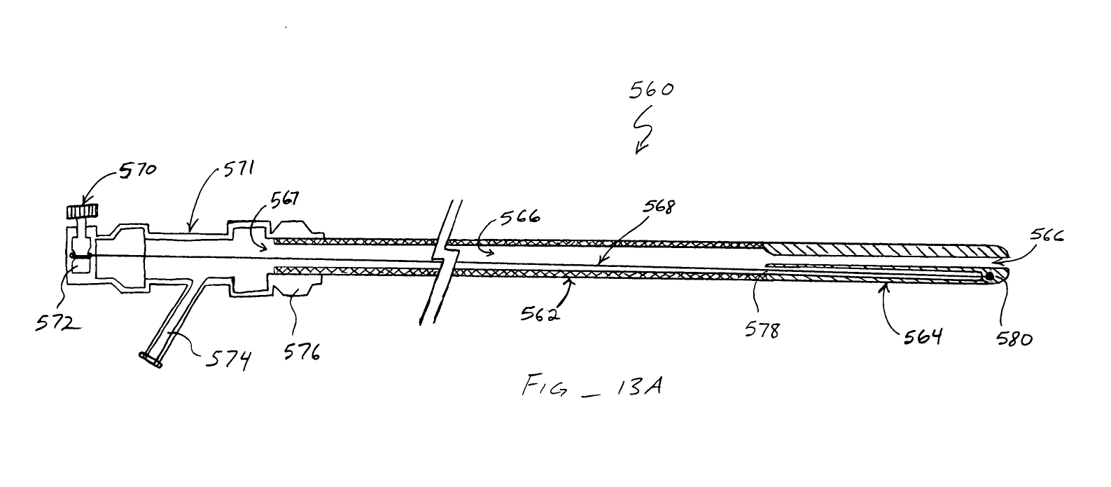

This can be more easily understood with reference to FIGS. 12A-12F, which demonstrate a method for advancing an anchor delivery device to a position for treating a mitral valve Mv. The mitral valve MV, including mitral valve leaflets MVL are represented diagrammatically from an inferior perspective looking up, to depict a method for delivering a device into subannular space 552. In FIG. 12A, first guide catheter 550 is show extending up to or into subannular space 552, as in FIG. 11. As shown in FIG. 12B, in one method a second guide catheter 554 may be advanced through first guide catheter 550 to pass through/along subannular space 554. This second guide catheter 554 is steerable in one embodiment, as will be described further below, to help conform second guide catheter 554 to subannular space 552.

Next, as in FIG. 12C, a guide sheath 556 may be passed over second guide catheter 554 to extend along subannular space. Sheath 556 is generally a flexible, tubular member that can be passed over second guide catheter 554 and within first guide catheter 550. To enhance passage and exchange, any of these and other described catheter members, sheath members, or the like may be manufactured from and/or coated with one or more friction resistant materials. Once sheath 556 is in place, second guide catheter 554 may be withdrawn, as shown in FIG. 12D. As shown in FIG. 12E, an anchor delivery device 558 may then be advanced through sheath 556 to a position for treating the mitral valve MV. Sheath 556 may then be withdrawn, as in FIG. 12F, leaving anchor delivery device 558 in place for performing a treatment. A valve annulus treatment may be performed, as described extensively above, and anchor delivery device 558 may be withdrawn. In some embodiments, anchor delivery device 558 is used to treat one portion of the valve annulus and is then moved to another portion, typically the opposite side, to treat the other portion of the annulus. In such embodiments, any one or more of the steps just described may be repeated. In some embodiments, anchor delivery device 558 is withdrawn through first guide catheter 550, and first guide catheter 550 is then withdrawn. In alternative embodiments, first guide catheter 550 may be withdrawn before anchor delivery device 558.

In various embodiments, alternative means may be used to urge anchor delivery device 558 into contact with the valve annulus. For example, in one embodiment an expandable member is coupled with anchor delivery device 558 and expanded within the subannular space 552. In an alternative embodiment, a magnet may be coupled with anchor delivery device 558, and another anchor may be disposed within the coronary sinus, in proximity to the first magnet. The two magnets may attract one another, thus pulling the anchor delivery device 558 into greater contact with the annulus. These or other embodiments may also include visualizing the annulus using a visualization member coupled with the anchor delivery device 558 or separate from the device 558. In some embodiments, anchors may be driven through a strip of detachable, biocompatible material, such as Dacron, that is coupled with anchor delivery device 558 but that detaches to affix to the valve annulus via the anchors. In some embodiments, the strip may then be cinched to tighten the annulus. In other embodiments, the anchors may be driven through a detachable, biocompatible, distal portion of the guide sheath 556, and guide sheath 556 may then remain attached to the annulus via the anchors. Again, in some embodiments, the detached sheath may be cinched to tighten the annulus.

Of course, the method just described is but one embodiment of a method for delivering an anchor delivery device to a location for treating a valve annulus. In various alternative embodiments, one or more steps may be added, deleted or modified while achieving a similar result. In some embodiments, a similar method may be used to treat the mitral valve from a superior/right atrial position or to treat another heart valve. Additionally, other devices or modifications of the system just described may be used in other embodiments.

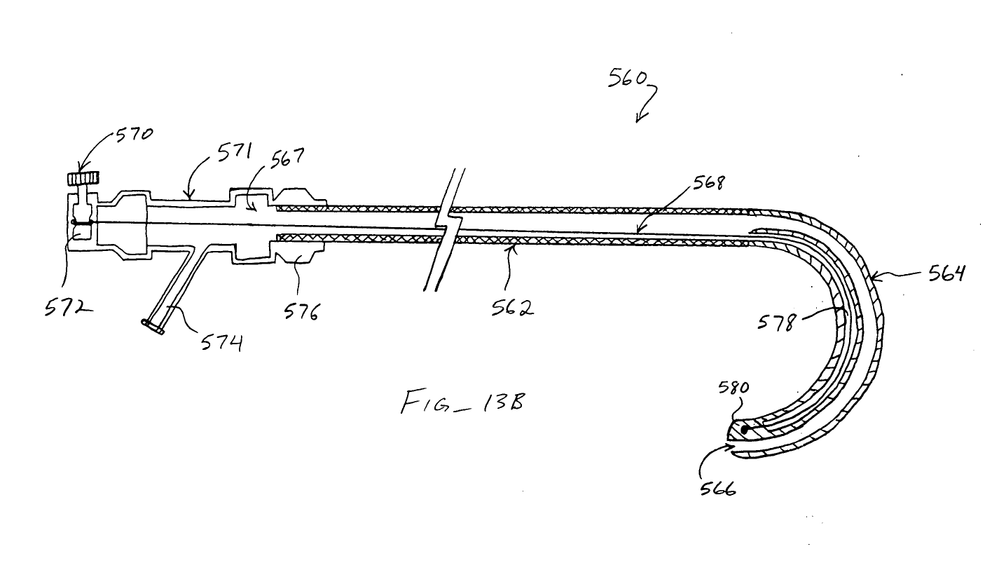

With reference now to FIGS. 13A and 13B, one embodiment of a steerable catheter device 560 is shown. Steerable catheter device 560 may be used in a method such as that just described in reference to FIGS. 12A-12F, for example in performing a function similar to that performed by second guide catheter 554. In other embodiments, catheter device 560 may perform any other suitable finction. As shown, catheter device 560 suitably includes an elongate catheter body having a proximal portion 562 and a distal portion 564. At least one tensioning member 568, such as but not limited to a tensioning cord, extends from proximal portion 562 to distal portion 564 and is coupled with the distal portion 564 and at least one tensioning actuator 570/572 on the proximal portion. Tensioning actuator 570/572 may include, for example, a knob 570 and a barrel 572 for wrapping and unwrapping tensioning member 568 to apply and remove tension. Tensioning member 568 is coupled with distal portion 564 at one or more connection points 580. In some embodiments, catheter device 560 includes a proximal housing 571, handle or the like, coupled to the proximal end of proximal portion 562 via a hub 576 or other means. Housing 571 may be coupled with tensioning actuator 570/572 and may include one or more arms 574 for infusing fluid or for other functions. In the embodiment shown, arm 574 and housing 571 include a lumen 567 that is in fluid communication with a fluid lumen 566 of the catheter body. Fluid may be introduced through arm 574 to pass through fluid lumen 566 to provide, for example, for contrast material at the distal tip of catheter device 560 to enhance visualization of device 560 during a procedure. Any other suitable fluid(s) may be passed through lumens 567/566 for any other purpose. Another lumen 578 may be included in distal portion 564, through which tensioning member 568 passes before attaching at a distal location along distal portion 564.

FIG. 13B shows catheter device 560 in a deformed/bent configuration, after tension has been applied to distal portion 564 by applying tension to tensioning member 568, via knob 570 and barrel 572. The bend in distal portion 564 will allow it to conform more readily to a valve annulus, while catheter device 560 in its straight configuration will be more amenable to passage through vasculature of the patient. Tensioning member 568 may be manufactured from any suitable material or combination of materials, such as but not limited to Nitinol, polyester, nylon, polypropylene and/or other polymers. Some embodiments may include two or more tensioning members 568 and/or two or more tensioning actuators 570/572 to provide for changes in shape of distal portion 564 in multiple directions. In alternative embodiments, knob 570 and barrel 572 may be substituted with any suitable devices, such as a pull cord, button, lever or other actuator. Various alternatives may also be substituted for tensioning member 568 in various embodiments. For example, shaped expandable members, shape memory members and/or the like may be used to change the shape of distal portion 564.

Generally, proximal portion 562 of the catheter body is less flexible than distal portion 564. Proximal portion 562 may be made of any suitable material, such as PEBAX, FEP, nylon, polyethylene and/or the like, and may include a braided material, such as stainless steel, to provide stiffness and strength. Distal portion 564 may be made of similar or other materials, but the braided material is typically not included, to provide for greater flexibility. Both proximal and distal portions 562/564 may have any suitable lengths, diameters, overall configurations and the like. In one embodiment the catheter body is approximately 140 cm in length and 6 French in diameter, but any other suitable sizes may be used in other embodiments. Either proximal portion 562, distal portion 564 or preferably both, may be made from or coated with one or more friction resistant or lubricating material to enhance passage of device 560 through an introducer catheter and/or to enhance passage of a sheath or other device over catheter device 560.

Although the foregoing is a complete and accurate description of the present invention, the description provided above is for exemplary purposes only, and variations may be made to the embodiments described without departing from the scope of the invention. Thus, the above described should not be construed to limit the scope of the invention as described in the appended claims.

What is claimed is:

1. A method for advancing one or more devices into a left ventricle of a heart to contact a mitral valve annulus, the method comprising:
advancing a steerable guide catheter into the left ventricle and around at least a portion of the mitral valve annulus;
passing a guide sheath over the steerable guide catheter;
withdrawing the steerable guide catheter out of the guide sheath; and
advancing one or more devices through the guide sheath to contact the mitral valve annulus.

2. A method as in claim 1, wherein the steerable guide catheter is advanced through an aorta into a space in the left ventricle formed by a left ventricular wall, at least one mitral valve leaflet and chordae tendiniae of the heart.

3. A method as in claim 2, further comprising deforming a flexible distal portion of the steerable guide catheter to conform the distal portion to the mitral valve annulus.

4. A method as in claim 3, wherein deforming the flexible distal portion comprises applying tension to at least one tensioning member to cause at least one bend in the distal portion.

5. A method as in claim 4, further comprising, before advancing the steerable guide catheter, advancing a shaped guide catheter through the aorta to a position within or adjacent the space in the left ventricle, wherein the steerable guide catheter is advanced through the shaped guide catheter.

6. A method as in claim 5, wherein deforming the flexible distal portion further comprises passing the distal portion through at least one bend in the shaped guide catheter.

7. A method as in claim 6, wherein passing the distal portion through the at least one bend in the shaped guide catheter comprises:
passing the portion through a first bend to direct it approximately into a plane with a plane of the mitral valve annulus; and
passing the portion through a second bend approximately perpendicular to the first bend and having a radius of curvature approximately the same as a radius of curvature of the mitral valve annulus.

8. A method as in claim 7, wherein applying tension to the at least one tensioning member causes the flexible distal portion to continue to bend in an arc with a radius of curvature approximately the same as the radius of curvature of the mitral valve annulus.

9. A method as in claim 4, wherein tension is applied to two tensioning members to articulate the flexible distal portion in at least two directions.

10. A method as in claim 3, wherein deforming the flexible distal portion comprises expanding a shaped expandable member to deform the distal portion.

11. A method as in claim 3, wherein deforming the flexible distal portion comprises introducing a fluid into a lumen of the distal portion.

12. A method as in claim 3, wherein deforming the flexible distal portion comprises releasing a shape-memory material from constraint.

13. A method as in claim 3, wherein deforming the flexible distal portion comprises articulating the distal portion in at least two directions.

14. A method as in claim 3, further comprising locking the shape of the flexible distal portion.

15. A method as in claim 3, further comprising urging the steerable guide catheter against the mitral valve annulus.

16. A method as in claim 15, wherein urging the steerable guide catheter comprises expanding an expandable member coupled with the steerable guide catheter within a space in the left ventricle formed by a left ventricular wall, at least one mitral valve leaflet and chordae tendiniae of the heart.

17. A method as in claim 15, wherein urging the steerable guide catheter comprises applying an attractive magnetic force between a first magnetic member coupled with the steerable guide catheter and a second magnetic member disposed within a coronary sinus of the heart.

18. A method as in claim 1, further comprising urging the guide sheath against the mitral valve annulus.

19. A method as in claim 18, wherein urging the guide sheath comprises expanding an expandable member coupled with the guide sheath within a space in the left ventricle formed by a left ventricular wall, at least one mitral valve leaflet and chordae tendiniae of the heart.

20. A method as in claim 18, wherein urging the guide sheath comprises applying an attractive magnetic force between a first magnetic member coupled with the guide sheath and a second magnetic member disposed within a coronary sinus of the heart.

21. A method as in claim 1, wherein a delivery device is advanced through the guide sheath for contacting and delivering a therapy to the mitral valve annulus.

22. A method as in claim 21, wherein the delivery device comprises a device for delivering coupled anchors to the mitral valve annulus, the method further comprising:
delivering a plurality of coupled anchors from the anchor delivery device to secure the anchors to the mitral valve annulus; and
drawing the anchors together to circumferentially tighten the annulus.

23. A method as in claim 22, further comprising expanding an expandable member coupled with the anchor delivery device to urge the delivery device against the length of valve annulus.

24. A method as in claim 22, further comprising applying an attractive magnetic force between a first magnetic member coupled with the delivery device and a second magnetic member disposed within a coronary sinus of the heart to urge the delivery device against the length of valve annulus.

25. A method as in claim 22, wherein the anchors are delivered from the anchor delivery device through a distal portion of the guide sheath to attach the distal portion to the mitral valve annulus, wherein the distal portion of the guide sheath is detachable from a proximal portion of the guide sheath to remain attached to the annulus.

26. A method as in claim 25, further comprising cinching the attached distal portion of the guide sheath to circumferentially tighten the valve annulus.

27. A method as in claim 22, wherein the anchors are delivered from the anchor delivery device through a detachable, biocompatible strip coupled with the anchor delivery device to attach the strip to the mitral valve annulus.

28. A method as in claim 27, further comprising cinching the attached strip to circumferentially tighten the valve annulus.

29. A method as in claim 22, further comprising:
contacting a stabilizing member with the valve annulus on a side of the valve opposite the anchor delivery device; and
applying force to the stabilizing member to immobilize the annulus between the stabilizing member and the anchor delivery device to facilitate delivery of the anchors.

30. A method as in claim 22, further comprising stabilizing the annulus with the anchor delivery device prior to delivering the anchors.

31. A method as in claim 30, wherein stabilizing the annulus with the anchor delivery device comprises expanding an expandable member coupled with the anchor delivery device to move the delivery device into enhanced contact with the valve annulus and stabilize the annulus.

32. A method as in claim 22, wherein the delivering and drawing steps cause a first length of the valve annulus to be tightened, the method further comprising:
contacting the anchor delivery device with a second length of the valve annulus;
delivering a plurality of coupled anchors from the anchor delivery device to secure the anchors to the second length of the annulus; and
drawing the anchors together to circumferentially tighten the second length of the annulus.

33. A method as in claim 21, further comprising delivering energy from the delivery device to tighten the valve annulus.

34. A method as in claim 33, wherein delivering energy comprises delivering a form of energy selected from the group consisting of radio frequency, ultrasound, microwave and laser energy.

35. A method as in claim 21, further comprising delivering at least one pharmacological agent from the delivery device to tighten the valve annulus.

36. A method as in claim 1, wherein a visualization device is advanced through the guide sheath for enhancing visualization of the mitral valve annulus.

37. A method as in claim 36, wherein the visualization device is selected from the group consisting of an ultrasound device, a camera, an endoscope and a fiber optic device.

38. A method as in claim 1, wherein all steps are performed while the heart is beating.

39. A method for advancing one or more devices into a left ventricle of a heart to contact a mitral valve annulus, the method comprising:
advancing a shaped guide catheter through an aorta into the left ventricle;
passing a steerable guide catheter through the shaped guide catheter and around at least a portion of the length of the mitral valve annulus;
passing a guide sheath over the steerable guide catheter, within the shaped guide catheter;
withdrawing the steerable guide catheter out of the guide sheath; and
advancing one or more devices through the guide sheath to contact the mitral valve annulus.

40. A method as in claim 39, wherein the steerable guide catheter is advanced into a space in the left ventricle formed by a left ventricular wall, at least one mitral valve leaflet and chordae tendiniae of the heart.

41. A method as in claim 39, further comprising deforming a flexible distal portion of the steerable guide catheter to conform the distal portion to the mitral valve annulus.

42. A method as in claim 41, wherein deforming the flexible distal portion comprises applying tension to at least one tensioning member to cause at least one bend in the distal portion.

43. A method as in claim 42, wherein deforming the flexible distal portion further comprises passing the distal portion through at least one bend in the shaped guide catheter.

44. A method as in claim 43, wherein passing the distal portion through the at least one bend in the shaped guide catheter comprises:
passing the portion through a first bend to direct it approximately into a plane with a plane of the mitral valve annulus; and
passing the portion through a second bend approximately perpendicular to the first bend and having a radius of curvature approximately the same as a radius of curvature of the mitral valve annulus.

45. A method as in claim 44, wherein applying tension to the at least one tensioning member causes the flexible distal portion to continue to bend in an arc with a radius of curvature approximately the same as the radius of curvature of the mitral valve annulus.

46. A method as in claim 42, wherein tension is applied to two tensioning members to articulate the flexible distal portion in at least two directions.

47. A method as in claim 41, wherein deforming the flexible distal portion comprises expanding a shaped expandable member to deform the distal portion.

48. A method as in claim 41, wherein deforming the flexible distal portion comprises introducing a fluid into a lumen of the distal portion.

49. A method as in claim 41, wherein deforming the flexible distal portion comprises releasing a shape-memory material from constraint.

50. A method as in claim 41, wherein deforming the flexible distal portion comprises articulating the distal portion in at least two directions.

51. A method as in claim 41, further comprising locking the shape of the flexible distal portion.

52. A method as in claim 41, further comprising urging the steerable guide catheter against the mitral valve annulus.

53. A method as in claim 52, wherein urging the steerable guide catheter comprises expanding an expandable member coupled with the steerable guide catheter within a space in the left ventricle formed by a left ventricular wall, at least one mitral valve leaflet and chordae tendiniae of the heart.

54. A method as in claim 52, wherein urging the steerable guide catheter comprises applying an attractive magnetic force between a first magnetic member coupled with the steerable guide catheter and a second magnetic member disposed within a coronary sinus of the heart.

55. A method as in claim 39, further comprising urging the guide sheath against the mitral valve annulus.

56. A method as in claim 55, wherein urging the guide sheath comprises expanding an expandable member coupled with the guide sheath within a space in the left ventricle formed by a left ventricular wall, at least one mitral valve leaflet and chordae tendiniae of the heart.

57. A method as in claim 55, wherein urging the guide sheath comprises applying an attractive magnetic force between a first magnetic member coupled with the guide sheath and a second magnetic member disposed within a coronary sinus of the heart.

58. A method as in claim 39, wherein a delivery device is advanced through the guide sheath for contacting and delivering a therapy to the mitral valve annulus.

59. A method as in claim 58, wherein the delivery device comprises a device for delivering coupled anchors to the mitral valve annulus, the method further comprising:
delivering a plurality of coupled anchors from the anchor delivery device to secure the anchors to the mitral valve annulus; and
drawing the anchors together to circumferentially tighten the annulus.

60. A method as in claim 59, further comprising expanding an expandable member coupled with the anchor delivery device to urge the delivery device against the length of valve annulus.

61. A method as in claim 59, further comprising applying an attractive magnetic force between a first magnetic member coupled with the delivery device and a second magnetic member disposed within a coronary sinus of the heart to urge the delivery device against the length of valve annulus.

62. A method as in claim 59, wherein the anchors are delivered from the anchor delivery device through a distal portion of the guide sheath to attach the distal portion to the mitral valve annulus, wherein the distal portion of the guide sheath is detachable from a proximal portion of the guide sheath to remain attached to the annulus.

63. A method as in claim 62, further comprising cinching the attached distal portion of the guide sheath to circumferentially tighten the valve annulus.

64. A method as in claim 59, wherein the anchors are delivered from the anchor delivery device through a detachable, biocompatible strip coupled with the anchor delivery device to attach the strip to the mitral valve annulus.

65. A method as in claim 64, further comprising cinching the attached strip to circumferentially tighten the valve annulus.

66. A method as in claim 59, further comprising:
contacting a stabilizing member with the valve annulus on a side of the valve opposite the anchor delivery device; and
applying force to the stabilizing member to immobilize the annulus between the stabilizing member and the anchor delivery device to facilitate delivery of the anchors.

67. A method as in claim 59, further comprising stabilizing the annulus with the anchor delivery device prior to delivering the anchors.

68. A method as in claim 59, wherein the delivering and drawing steps cause a first length of the valve annulus to be tightened, the method further comprising:
contacting the anchor delivery device with a second length of the valve annulus;
delivering a plurality of coupled anchors from the anchor delivery device to secure the anchors to the second length of the annulus; and
drawing the anchors together to circumferentially tighten the second length of the annulus.

69. A method as in claim 58, further comprising delivering energy from the delivery device to tighten the valve annulus.

70. A method as in claim 69, wherein delivering energy comprises delivering a form of energy selected from the group consisting of radio frequency, ultrasound, microwave and laser energy.

71. A method as in claim 58, further comprising delivering at least one pharmacological agent from the delivery device to tighten the valve annulus.

72. A method as in claim 39, wherein a visualization device is advanced through the guide sheath for enhancing visualization of the mitral valve annulus.

73. A method as in claim 72, wherein the visualization device is selected from the group consisting of an ultrasound device, a camera, an endoscope and a fiber optic device.

74. A method as in claim 39, wherein all steps are performed while the heart is beating.

75. A method for treating a mitral valve annulus of a heart, the method comprising:
advancing a steerable guide catheter into a left ventricle of the heart and around at least a portion of the mitral valve annulus;
passing a guide sheath over the steerable guide catheter;
withdrawing the steerable guide catheter out of the guide sheath;
advancing an anchor delivery device through the guide sheath to contact the mitral valve annulus;
delivering a plurality of coupled anchors from the anchor delivery device to secure the anchors to the mitral valve annulus; and
drawing the anchors together to circumferentially tighten the annulus.

76. A method as in claim 75, further comprising, before advancing the steerable guide catheter, advancing a shaped guide catheter through the aorta to a position within or adjacent the space in the left ventricle, wherein the steerable guide catheter is advanced through the shaped guide catheter.

77. A device for facilitating placement of one or more devices in contact with a heart valve annulus, the device comprising:
an elongate catheter body having a proximal portion and a distal portion;
at least one tensioning member coupled with the proximal portion of the catheter body and extending to the distal portion; and
at least one tensioning actuator coupled with the proximal portion and the tensioning member for applying tension to the tensioning member to deform the distal portion to allow it to conform generally to a shape of the valve annulus.

78. A device as in claim 77, wherein the catheter body may be advanced intravascularly to the heart to contact the annulus.

79. A device as in claim 78, wherein the catheter body may be advanced through an aorta and into a left ventricle of the heart to contact the valve annulus.

80. A device as in claim 78, wherein the proximal portion of the catheter body is relatively stiff compared to the distal portion.

81. A device as in claim 78, wherein the catheter body further comprises a rounded, atraumatic distal tip.

82. A device as in claim 81, wherein the catheter body further comprises at least one radiopaque portion at or near the distal tip for enhancing visualization.

83. A device as in claim 77, wherein the catheter body further comprises at least one lumen extending through the proximal and distal portions for passing one or more fluids.

84. A device as in claim 77, wherein the at least one tensioning member comprises two tensioning members, allowing the distal portion to be deformed in at least two different directions.

85. A device as in claim 77, wherein the at least one tensioning member comprises at least one tensioning cord comprising a material selected from the group consisting of Nitinol, polyester, nylon, polypropylene and other polymers.

86. A device as in claim 77, wherein the at least one tensioning actuator comprises a knob coupled with the tensioning member, wherein turning the knob in one direction applies tension to the tensioning member to deform the distal portion, and wherein turning the knob in an opposite direction releases tension from the tensioning member to return to the distal portion to a less deformed configuration.

87. A device as in claim 77, further comprising at least one urging member coupled with the distal portion of the catheter body for urging the distal portion into contact with the valve annulus.

88. A device as in claim 87, wherein the at least one urging member comprises an expandable member for expanding within a space in a left ventricle formed by a left ventricular wall, at least one mitral valve leaflet and chordae tendiniae of the heart.

89. A device as in claim 87, wherein the at least one urging member comprises at least one magnet coupled with the distal portion for applying attractive magnetic force between itself and an oppositely charged magnet disposed in a coronary sinus adjacent the valve annulus.

90. A device as in claim 77, further comprising a housing coupled with the proximal end, wherein the tensioning actuator is coupled with the housing.

91. A device as in claim 90, wherein the housing further comprises at least one fluid inlet port in fluid communication with at least one lumen in the elongate shaft for introducing one or more fluids into the lumen(s).

92. A system for facilitating placement of one or more devices in contact with a heart valve annulus, the system comprising:
a shaped guide catheter having at least one curve toward a distal end for positioning the distal end in a position below the mitral valve;
a steerable guide catheter passable through the shaped guide catheter and having a steerable distal end for advancing around a length of the valve annulus below the mitral valve; and
a guide sheath passable over the steerable guide catheter through the shaped guide catheter, wherein the one or more devices are passable through the guide sheath to contact the mitral valve annulus.

93. A system as in claim 92, wherein the at least one curve of the shaped guide catheter comprises:
a proximal curve approximately perpendicular to a central axis of the shaped guide catheter for bringing the distal end of the catheter into a plane approximately parallel with a plane of the mitral valve; and
a distal curve having a radius of curvature approximately the same as a radius of curvature of the mitral valve annulus.

94. A system as in claim 92, wherein the steerable guide catheter comprises:
an elongate catheter body having a proximal portion and a distal portion;
at least one tensioning member coupled with the proximal portion of the catheter body and extending to the distal portion; and
at least one tensioning actuator coupled with the proximal portion and the tensioning member for applying tension to the tensioning member to deform the distal portion to allow it to conform generally to a shape of the valve annulus.

95. A system as in claim 94, wherein the catheter body may be advanced intravascularly to the heart to contact the annulus.

96. A system as in claim 95, wherein the catheter body may be advanced through an aorta and into a left ventricle of the heart to contact the valve annulus.

97. A system as in claim 95, wherein the proximal portion of the catheter body is stiffer than the distal portion.

98. A system as in claim 95, wherein the catheter body further comprises a rounded, atraumatic distal tip.

99. A system as in claim 98, wherein the catheter body further comprises at least one radiopaque portion at or near the distal tip for enhancing visualization.

100. A system as in claim 94, wherein the catheter body further comprises at least one lumen extending through the proximal and distal portions for passing one or more fluids.

101. A system as in claim 94, wherein the at least one tensioning member comprises two tensioning members, allowing the distal portion to be deformed in at least two different directions.

102. A system as in claim 94, wherein the at least one tensioning member comprises at least one tensioning cord comprising a material selected from the group consisting of Nitinol, polyester, nylon, polypropylene and other polymers.

103. A system as in claim 94, wherein the at least one tensioning actuator comprises a knob coupled with the tensioning member, wherein turning the knob in one direction applies tension to the tensioning member to deform the distal portion, and wherein turning the knob in an opposite direction releases tension from the tensioning member to return to the distal portion to a less deformed configuration.

104. A system as in claim 94, further comprising at least one urging member coupled with the distal portion of the catheter for urging the distal portion into contact with the valve annulus.

105. A system as in claim 104, wherein the at least one urging member comprises an expandable member for expanding within a space in a left ventricle formed by a left ventricular wall, at least one mitral valve leaflet and chordae tendiniae of the heart.

106. A system as in claim 104, wherein the at least one urging member comprises at least one magnet coupled with the distal portion for applying attractive magnetic force between itself and an oppositely charged magnet disposed in a coronary sinus adjacent the valve annulus.

107. A system as in claim 94, further comprising a housing coupled with the proximal end, wherein the tensioning actuator is coupled with the housing.

108. A system as in claim 107, wherein the housing further comprises at least one fluid inlet port in fluid communication with at least one lumen in the elongate shaft for introducing one or more fluids into the lumen(s).

109. A system as in claim 92, wherein a distal portion of the guide sheath is detachable from a proximal portion of the guide sheath to remain in attached to the valve annulus after an annulus treatment procedure.

110. A system as in claim 109, wherein the detachable distal portion comprises a tubular member comprising Dacron.

111. A system as in claim 109, wherein the detachable distal portion is cinchable to tighten the mitral valve annulus.

112. A system as in claim 92, further comprising at least one urging member coupled with at least one of the shaped guide catheter, the steerable guide catheter and the guide sheath.

113. A device as in claim 112, wherein the at least one urging member comprises an expandable member for expanding within a space in a left ventricle formed by a left ventricular wall, at least one mitral valve leaflet and chordae tendiniae of the heart.

114. A device as in claim 112, wherein the at least one urging member comprises at least one magnet coupled with at least one of the shaped guide catheter, the steerable guide catheter and the guide sheath for applying attractive magnetic force between itself and an oppositely charged magnet disposed in a coronary sinus adjacent the valve annulus.

115. A system as in claim 92, further comprising an anchor delivery device passable through the guide sheath contact and apply coupled anchors to the mitral valve annulus.

116. A system as in claim 92, further comprising a visualization device passable through the sheath guide to facilitate visualization of the mitral valve annulus.

117. A system as in claim 116, wherein the visualization device is selected from the group consisting of an ultrsound device, a camera, an endoscope and a fiber optic device.
